(19) United States
(12) Patent Application Publication
(10) Pub. No.: US 2025/0228692 A1
(43) Pub. Date: Jul. 17, 2025

(54) SUPPORT SURFACE OVERLAY WITH USER RETENTION FEATURES

(71) Applicant: IPLEXXUS HOLDINGS, LLC, Freeland, MI (US)

(72) Inventor: David A. DZIOBA, Freeland, MI (US)

(21) Appl. No.: 19/021,989

(22) Filed: Jan. 15, 2025

Related U.S. Application Data
(60) Provisional application No. 63/558,831, filed on Feb. 28, 2024.
(60) Provisional application No. 63/621,304, filed on Jan. 16, 2024.

(51) Int. Cl. A61F 5/34; A61F 5/37

(52) U.S. Cl. CPC A61F 5/34; A61F 5/3776

(57) ABSTRACT

A support surface overlay includes a selectively inflatable compartment including a plurality of selectively inflatable pivoting elements configured to pivot with respect to adjacent portions of the overlay when the selectively inflatable compartment is inflated and deflated. The pivoting elements are configured and operable to selectively apply and release shear forces to and from the skin of a user lying upon the overlay when the selectively inflatable compartment is inflated and deflated. The support surface overlay may include a body retention feature configured to hinder lateral displacement along or across a body-supporting surface of the support surface overlay in response to inflation and deflation of the selectively inflatable compartment or otherwise. The body retention feature may be a body retention strap including a selectively inflatable pivoting element configured to pivot with respect to an adjacent portion of the body retention strap when the selectively inflatable pivoting element is inflated and deflated. The body retention feature may be a deformable layer or inflatable bolster that envelopes a portion of a user lying on the support surface overlay and thereby resists slippage of the user.

### CROSS-REFERENCE TO RELATED APPLICATIONS

This application claims benefit under 35 U.S.C. § 119 of U.S. Provision Patent Application No. 63/621,304, filed on Jan. 16, 2024, and U.S. Provision Patent Application No. 63/558,831, filed on Feb. 28, 2024. This application incorporates by reference the disclosure of the foregoing applications in their entireties.

### BACKGROUND OF THE DISCLOSURE

Decubitus ulcers, commonly referred to as pressure ulcers, may result from prolonged occlusion of capillary blood flow in human skin tissue. One factor that contributes to occlusion of capillary blood flow is application of contact pressure to human skin (sometimes referred to as interface pressure) in excess of the vascular occlusion threshold, which commonly is regarded to be about 28-32 mm Hg. Such contact pressure may be applied to the skin, for example, in a direction having a component normal to the skin in response to the force of gravity applied to a human lying on a support surface, for example, a hospital bed, an operating table, or the like.

Another factor that contributes to occlusion of capillary blood flow is application of shear force to the skin (sometimes referred to as skin shear). Shear force may be applied to the skin, for example, by a surface pulling the skin in a direction having a component generally parallel to the skin through frictional engagement with the skin. For example, the force of gravity acting upon the skin of a user disposed on an inclined support surface has a first directional component normal to the skin and a second directional component parallel to the skin. Absent frictional engagement of the skin with the support surface, either directly or through one or more intervening layers, for example, bed sheets, the parallel force component would cause the user to slide off of the support surface. Put another way, frictional engagement of the skin with the support surface counteracts the parallel force component and prevents the user from sliding off of the support surface. In doing so, the frictional engagement force pulls the user's skin in a direction generally parallel to the skin, and thus contributes to occlusion of capillary blood flow in the frictionally-engaged region of the skin.

Support surfaces and support surface overlays intended to remediate and inhibit formation of decubitus ulcers are known in the art. For example, traditional support surfaces and techniques seek to distribute a user's weight over a relatively large area (typically as large an area as possible) so that the interface pressure between the support surface and the user's body generally remains below the vascular occlusion threshold. Such support surfaces and techniques are sometimes referred to as “redistribution” surfaces and techniques. Known redistribution support surfaces and techniques may involve relatively thick air mattresses having alternatingly inflatable compartments defining relatively large air cells that are operated at relatively low internal pressures. In an embodiment, a traditional redistribution surface may be several inches thick when inflated, have air cells several inches in diameter, and be operated at internal pressures of about 0.5-1.0 psig. Although their use is widespread, such traditional support surfaces and techniques are imperfect and often fail to provide adequate relief from vascular occlusion.

Another approach involves a support surface overlay substantially thinner than a typical redistribution surface and having substantially smaller air cells operated at substantially higher pressures. For example, such a support surface overlay may be about 1.0 inch thick when inflated, may have alternatingly inflatable air cells (or lifting nodes) about 1.0 inch in diameter, and may be operated at internal pressures of about 4.0 psig or greater. Such a support surface overlay may enhance perfusion by virtue of the lifting nodes alternatingly and temporarily applying controlled contact pressure to portions of the user's body in excess of the vascular occlusion threshold, while at the same time reducing contact pressure below the vascular occlusion threshold at interstices between the lifting nodes. Such a support surface overlay typically requires the use of an underlying redistribution layer in the form of a redistribution surface or pad to avoid bottoming out from the user's weight, particularly in the area of bony prominences. In such embodiments, the redistribution layer provides average pressure redistribution for the entire user contact area, while the lifting nodes of the support surface overlay provide localized alternating support and pressure relief, along with relief of shear forces imparted to the user's skin further to initial positioning of the user on the support surface overlay.

Traditionally, application of shear forces to the skin has been regarded as a problem to be avoided to the extent practical, and intentional application of shear forces to the skin has not been embraced as a technique for enhancing perfusion. Indeed, some known support surface overlays seek to reduce skin shear resulting from expansion and contraction of inflatable compartments thereof, for example, by employing relief cuts to decouple some lifting nodes thereof from other lifting nodes thereof to thereby mitigate application of shear forces that may be applied to the skin further to relative side-to-side motion of the lifting nodes in response to inflation and deflation thereof.

Such support surface overlays and techniques, however, may encourage lateral displacement or “walking” of a user along or across the surface of the support surface overlay, especially when in an inclined orientation, that is, an orientation other than horizontal. Such walking may occur because a component of gravitational force parallel to the support surface overlay and user tends to pull the user toward a lower extent of the user-bearing surface of the support surface overlay. Friction between the user and the lifting nodes of a first, inflatable compartment of the support surface overlay when such lifting nodes are inflated and in body-supporting contact with the user tends to skew the inflated lifting node in the same direction. When a second compartment of the support surface overlay having lifting nodes adjacent the lifting nodes of the first inflatable compartment is then inflated and the first inflatable compartment is deflated, friction between the user and the lifting nodes of the first inflatable compartment is relieved, allowing the user to incrementally move toward the lower extent of the user-bearing surface of the support surface overlay. Such incremental movement may continue with each incidence of alternating inflation and deflation of the inflatable compartments of the support surface overlay.

Some conventional systems and techniques use straps to hinder such walking or to otherwise retain a user on a support surface or support surface overlay, especially when inclined. Such straps, however, can apply contact pressure and shear forces to portions of the user's skin engaged by the straps sufficient to locally occlude capillary blood flow. Such contact pressure and shear forces may remain unrelieved for a substantial portion of the duration of, for example, a surgical procedure, which may take hours to complete. As mentioned above, such unrelieved application of contact pressure and shear force to the skin for an extended period of time may lead to formation of decubitus ulcers.

### SUMMARY

The present disclosure is directed to support surface overlays including selectively operable pivoting contact elements that may apply and release shear forces to and from skin of a user lying upon such support surface overlays in a controlled manner. Such support surface overlays also may include body retention features, for example, body retention straps configured to wrap around at least a portion of the user's body. Such body retention straps also may include such pivoting contact elements. Such controlled application and release of shear forces to and from the skin further to operation of the pivoting contact elements may enhance perfusion in the regions of the skin subject to such application and release of shear forces.

In embodiments, a support surface overlay according to the present disclosure has a bladder including a first flat, flexible sheet, a second flat, flexible sheet overlying the first flat, flexible sheet; and a seam joining the first, flat flexible sheet to the second flat, flexible sheet at predetermined locations. The support surface overlay also may include a body retention strap operably associated with the bladder, the body retention strap including a selectively inflatable strap pivoting element, wherein the selectively inflatable strap pivoting element is inflatable and deflatable between a deflated state and an inflated state, and wherein the selectively inflatable strap pivoting element is configured to pivot relative to adjacent portions of the body retention strap in response to transition of the strap pivoting element between an deflated state and an inflated state.

In embodiments, the body retention strap may be configured to wrap around at least a portion of a body received upon the overlay. In embodiments, the body retention strap may be an upper body strap configured to wrap around at least a portion of an upper portion of a body received upon the overlay. In embodiments, the body retention strap may be a shoulder strap configured to wrap around at least a portion of a shoulder of a body received upon the overlay. In embodiments, the body retention strap may be configured to wrap around at least a portion of an arm of a body received upon the overlay or an underlying support surface. In embodiments, the body retention strap may be monolithically formed with a portion of the support surface overlay. In embodiments, the body retention strap may be monolithically formed with the bladder.

In embodiments, the strap pivoting element may be fluidly connected to the first selectively inflatable compartment via a fluid conduit internal to the bladder. In embodiments, the strap pivoting element may be fluidly connected to the first selectively inflatable compartment via a fluid conduit external to the bladder. In embodiments, the strap pivoting element may be formed separately from the bladder. In embodiments, the strap pivoting element may be fluidly connected to the first inflatable compartment.

In embodiments, the support surface overlay may further include a flexible and resilient deformable layer integrated with the bladder, wherein the deformable layer may be configured to flexibly and resiliently deform under the weight of a body disposed upon the overlay. In embodiments, the deformable layer may define at least one preformed depression.

In embodiments, the support surface overlay may further include a selectively inflatable bolster compartment configured to at least partially envelope a region of a body of a user disposed upon the support surface overlay. In embodiments, the region of the user may be a shoulder region or upper body region.

In embodiments, the support surface overlay may further include a separation cut proximate the selectively inflatable bolster compartment, wherein the separation cut is configured to lift of the selectively inflatable bolster compartment with respect to adjacent portions of the support surface overlay.

The present disclosure also is directed to a body retention strap for use in connection with the support surface overlay, wherein the body retention strap includes a selectively inflatable strap pivoting element, wherein the selectively inflatable strap pivoting element is inflatable and deflatable between a deflated state and an inflated state, and wherein the selectively inflatable strap pivoting element is configured to pivot relative to adjacent portions of the body retention strap in response to transition of the strap pivoting element between an deflated state and an inflated state.

In embodiments, a support surface overlay system according to the present disclosure may include a bladder having a first flat, flexible sheet, a second flat, flexible sheet overlying the first flat, flexible sheet, and a seam joining the first, flat flexible sheet to the second flat, flexible sheet at predetermined locations. The support surface overlay system also may include a flexible and resilient deformable layer integrated with the bladder, wherein the deformable layer is configured to flexibly and resiliently deform under the weight of a body disposed upon the overlay. In embodiments, the deformable layer may define at least one preformed depression.

The present disclosure further is directed to a body retention strap for use in connection with a support surface overlay, wherein the body retention strap includes a selectively inflatable strap pivoting element, wherein the selectively inflatable strap pivoting element is inflatable and deflatable between a deflated state and an inflated state, and wherein the selectively inflatable strap pivoting element is configured to pivot relative to adjacent portions of the body retention strap in response to transition of the strap pivoting element between an deflated state and an inflated state.

### BRIEF DESCRIPTION OF THE DRAWINGS

FIG. 1A is a top plan view of an illustrative support surface overlay having first and second selectively inflatable compartments according to the present disclosure;

FIG. 1B is an end elevation view of the support surface overlay of FIG. 1A disposed on an underlying support surface.

FIG. 1C is a top plan view of the support surface overlay of FIG. 1A with the second inflatable compartment removed to more clearly show the first inflatable compartment;

FIG. 1D is a top plan view of the support surface overlay of FIG. 1A with the first inflatable compartment removed to more clearly show the second inflatable compartment;

FIG. 1E is a front elevation view of a human skeleton showing skeletal structure in detail;

FIG. 1F is a bottom end view of an outline of the human skeleton of FIG. 1E with the detailed skeletal structure deleted for clarity;

FIG. 1G is a top plan view of the outline of the human skeleton of FIG. 1F lying upon the support surface overlay of FIG. 1A;

FIG. 1H is an end elevation view of the outline of the human skeleton and support surface overlay of FIG. 1G disposed upon an underlying support surface;

FIG. 2A is a top plan view of the support surface overlay of FIG. 1A with detail removed to better show first and second manifolds of the first and second selectively inflatable compartments;

FIG. 2B is an end elevation view of the support surface overlay as shown in FIG. 2A disposed on an underlying support surface;

FIG. 3A is a top plan view of the support surface overlay of FIG. 1A with detail removed to better show anchor regions thereof;

FIG. 3B is an end elevation view of the support surface overlay as shown in FIG. 3A disposed on an underlying support surface;

FIG. 4A is a detail view of the support surface overlay as shown in FIG. 3, further showing anchor straps for use in anchoring the support surface overlay to an underlying support surface;

FIG. 4B is a detail view of the support surface overlay as shown in FIG. 4A disposed on an underlying support surface;

FIG. 5 is a set of cross sectional views of a portion of the support surface overlay of FIG. 2 showing a sequence of assembling and processing first and second flat, flexible sheets to form the support surface overlay of FIG. 1A;

FIG. 6A is top plan detail view of a portion of the support surface overlay of FIG. 1A showing several selectively inflatable lifting elements thereof;

FIG. 6B is a cross-sectional view of the portion of the support surface overlay shown in FIG. 6A with the selectively inflatable lifting elements in an inflated state;

FIG. 7A is a set of cross-sectional elevation views of the support surface overlay of FIG. 5 showing a sequence of adding a protective sheet upon the second flat, flexible sheet;

FIG. 7B is a set of cross-sectional elevation views of the support surface overlay of FIG. 5 showing a sequence of adding a first envelope layer beneath the first flat, flexible sheet, and adding a second envelope layer upon the second flat, flexible sheet;

FIG. 8A is a top plan view detail view of the support surface overlay of FIG. 1A showing plural selectively inflatable lifting elements, separation cuts defining the shape of the lifting elements, anchor points at ends of the separation cuts, and anchor lines defined by the anchor points;

FIG. 8B is a top plan view detail view of the support surface overlay of FIG. 1A showing one of the selectively inflatable lifting elements and corresponding separation cuts and anchor points of FIG. 8A in greater detail;

FIG. 9A is a cross-sectional detail elevation views of the selectively inflatable lifting element of FIGS. 8A and 8B in inflated and deflated states;

FIG. 9B is a cross-sectional detail elevation views of one of the selectively inflatable lifting elements of FIGS. 8A and 8B transitioning between inflated and deflated states;

FIG. 10A is a top plan detail view of a selectively inflatable lifting element of the support surface overlay of FIG. 1A extending over a substantial portion of the width of the support surface overlay;

FIG. 10B is a top plan view of a pair of selectively inflatable lifting elements of the support surface overlay of FIG. 1A, each extending over about half the width of the selectively inflatable lifting element shown in FIG. 10A;

FIG. 10C is a top plan view of a pair of selectively inflatable lifting element of the support surface overlay of FIG. 1A similar to those shown in FIG. 10B, but with non-inflatable portions contained therein;

FIG. 11A is a top plan detail view of the support surface overlay of FIG. 1A showing plural selectively inflatable pivoting elements, separation cuts defining the shape of the pivoting elements, anchor points at ends of the separation cuts, and hinge lines defined by the anchor points;

FIG. 11B is a top plan detail view of the support surface overlay of FIG. 1A showing an alternative form of one of the selectively inflatable pivoting elements and corresponding separation cuts and anchor points;

FIG. 12A is a perspective view of a portion of the support surface overlay of FIG. 1A including a plurality of selectively pivoting elements thereof and showing how such pivoting elements may pivot relative to adjacent portions of the support surface overlay;

FIG. 12B is a cross-sectional detail elevation view of the selectively inflatable pivoting elements of FIGS. 11A and 11B in inflated and deflated states;

FIG. 12C is a cross-sectional detail elevation view of one of the selectively inflatable pivoting element of FIGS. 11A and 11B transitioning between inflated and deflated states and disposed on an underlying inclined support surface;

FIGS. 13A-13C are top plan detail views of alternative pivoting elements having alternative shapes;

FIG. 14A is a cross-sectional side elevation view of a slice of a portion of the support surface overlay of FIG. 1A disposed upon a horizontal support surface showing, in deflated states, two first lifting elements and one second lifting element defined by separation cuts decoupling the lifting elements from adjacent portions of the support surface overlay, wherein the slice is taken through the centers of the lifting elements;

FIG. 14B is a cross-sectional side elevation view similar to FIG. 14A but showing the first lifting elements in inflated states and the second lifting element in a deflated state, wherein the lifting elements would tend to lift a body (not shown) disposed thereon when transitioning from a deflated state to an inflated state;

FIG. 14C is a cross-sectional side elevation view similar to FIG. 14B but showing the support surface overlay disposed upon an underlying inclined support surface, and further showing that the lifting elements would have a tendency to pivot in a clockwise direction down the incline in response to the force of gravity when a body (not shown) is disposed on the lifting elements when transitioning from a deflated state to an inflated state;

FIG. 15A is a cross-sectional side elevation view of a slice of a portion of the support surface overlay of FIG. 1A disposed upon a horizontal support surface, showing two first lifting elements and one second lifting element not defined by separation cuts and in deflated states, wherein the slice is taken through the centers of the lifting elements;

FIG. 15B is a cross-sectional side elevation view similar to FIG. 15A but showing the first lifting elements in inflated states and the second lifting element in a deflated state, wherein the lifting elements would tend to lift a body (not shown) disposed thereon when transitioning from a deflated state to an inflated state;

FIG. 15C is a cross-sectional side elevation view similar to FIG. 15B but showing the support surface overlay disposed upon an underlying inclined support surface, and further showing that the lifting elements would have a tendency to pivot in a clockwise direction down the incline in response to the force of gravity when a body (not shown) is disposed on the lifting elements when transitioning from a deflated state to an inflated state;

FIG. 16A is a cross-sectional side elevation view of a slice of a portion of the support surface overlay of FIG. 1A disposed upon a horizontal support surface showing, in deflated states, two first pivoting elements and one second pivoting element defined by separation cuts decoupling the pivoting elements from adjacent portions of the support surface overlay, wherein the slice is taken through the centers of the pivoting elements;

FIG. 16B is a cross-sectional side elevation view similar to FIG. 16A but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface overlay to the left when transitioning from a deflated state to an inflated state;

FIG. 16C is a cross-sectional side elevation view similar to FIG. 16A, but showing the support surface overlay on an underlying inclined support surface;

FIG. 16D is a cross-sectional side elevation view similar to FIG. 16C but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface to the left and up the incline when transitioning from a deflated state to an inflated state;

FIG. 17A is a cross-sectional side elevation view of a slice of a portion of the support surface overlay of FIG. 1A disposed upon a horizontal support surface showing, in deflated states, two first pivoting elements and one second pivoting element not defined by separation cuts, wherein portions of the second sheet defining the respective pivoting elements are locally thinned compared to adjacent portions of the second sheet and compared to portions of the first sheet defining the respective pivoting elements, and wherein the slice is taken through the centers of the pivoting elements;

FIG. 17B is a cross-sectional side elevation view similar to FIG. 17A but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface overlay to the left when transitioning from a deflated state to an inflated state;

FIG. 17C is a cross-sectional side elevation view similar to FIG. 17A, but showing the support surface overlay on an underlying inclined support surface;

FIG. 17D is a cross-sectional side elevation view similar to FIG. 17C but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface to the left and up the incline when transitioning from a deflated state to an inflated state;

FIG. 18A is a cross-sectional side elevation view of a slice of a portion of the support surface overlay of FIG. 1A disposed upon a horizontal support surface showing, in deflated states, two first pivoting elements and one second pivoting element defined by separation cuts decoupling the pivoting elements from adjacent portions of the support surface overlay, wherein portions of the second sheet defining the respective pivoting elements are locally thinned compared to adjacent portions of the second sheet and compared to portions of the first sheet defining the respective pivoting elements, wherein the slice is taken through the centers of the pivoting elements;

FIG. 18B is a cross-sectional side elevation view similar to FIG. 18A but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface overlay to the left when transitioning from a deflated state to an inflated state;

FIG. 18C is a cross-sectional side elevation view similar to FIG. 18A, but showing the support surface overlay on an underlying inclined support surface;

FIG. 18D is a cross-sectional side elevation view similar to FIG. 18C but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface to the left and up the incline when transitioning from a deflated state to an inflated state;

FIG. 19A is a cross-sectional side elevation view of a slice of a portion of the support surface overlay of FIG. 1A disposed upon a horizontal support surface showing, in deflated states, two first pivoting elements and one second pivoting element defined by separation cuts decoupling the pivoting elements from adjacent portions of the support surface overlay, wherein the entirety of the second sheet defining the respective pivoting elements is thinner than the portions of the first sheet defining the respective pivoting elements, wherein the slice is taken through the centers of the pivoting elements;

FIG. 19B is a cross-sectional side elevation view similar to FIG. 19A but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface overlay to the left when transitioning from a deflated state to an inflated state;

FIG. 19C is a cross-sectional side elevation view similar to FIG. 19A, but showing the support surface overlay on an underlying inclined support surface;

FIG. 19D is a cross-sectional side elevation view similar to FIG. 19C but showing the first pivoting elements in inflated states and the second pivoting element in a deflated state, wherein both of the first pivoting elements have pivoted in a counterclockwise direction so that both of the two first pivoting elements would tend to translate at least a portion of body that might be disposed upon the support surface to the left and up the incline when transitioning from a deflated state to an inflated state;

FIG. 20 is a cross-sectional side view of an illustrative apparatus and method for forming the regions of reduced thickness of the upper sheet of the support surface overlay of FIGS. 17A-D and 18A-18D according to the present disclosure.

FIG. 21A is a top plan view of a portion of the support surface overlay of FIG. 1A showing pivoting elements defined by separation cuts outside anchor zones of the support surface overlay;

FIG. 21B is an end elevation view of the support surface overlay as shown in FIG. 21A;

FIG. 21C is a top plan detail view of a portion of FIG. 21A;

FIG. 21D is an end elevation view of the support surface overlay as shown in FIG. 21C;

FIG. 22A is a top plan view of a portion of the support surface overlay of FIG. 1A showing pivoting elements defined by separation cuts entering anchor zones of the support surface overlay and reinforced by anchor reinforcements;

FIG. 22B is an end elevation view of the support surface overlay as shown in FIG. 22A;

FIG. 22C is a top plan detail view of a portion of FIG. 22A;

FIG. 22D is an end elevation view of the support surface overlay as shown in FIG. 22C;

FIG. 23A is a top plan view of the support surface overlay of FIG. 1A further including monolithically-formed body retention straps and anchor straps configured to secure the support surface overlay to an underlying support surface, for example, an operating room table or a bed mattress according to the present disclosure, with the internal structure of the support surface overlay omitted for clarity;

FIG. 23B is a top plan view of the support surface overlay of FIG. 23A further including securement systems associated with ones of the body retention straps according to the present disclosure;

FIGS. 23C and 23D are similar to FIGS. 23A and 23B, respectively, but further show an outline of a human skeletal structure disposed upon the support surface overlay according to the present disclosure;

FIG. 23E is a top plan view of the support surface overlay of FIG. 23D showing the body retention straps securing the outline of the human skeletal structure to the support surface overlay;

FIG. 23F is an end elevation view of the support surface overlay and skeletal structure shown in FIG. 23F disposed upon an underlying support surface;

FIG. 23G is a detail view of a portion of the support surface overlay of FIG. 23E showing selectively inflatable strap pivoting elements and strap lifting elements disposed in ones of body retention straps thereof;

FIG. 24A is a front elevation view of a body retention strap apparatus including upper body straps and shoulder straps having strap pivoting elements and strap lifting elements according to the present disclosure;

FIG. 24B is a top plan view of the body retention strap apparatus of FIG. 24A in use strapping a human skeletal structure to an underlying support surface;

FIG. 24C is an end elevation view of the body retention strap apparatus, skeletal structure, and underlying support surface of FIG. 24B;

FIG. 25 is a top plan view of separately-formed upper body straps post-attached to the support surface of FIG. 1A according to the present disclosure;

FIG. 26A is a top plan view of the support surface overlay of FIG. 1A further including an integral deformable layer according to the present disclosure;

FIG. 26B is an end elevation view of the support surface overlay with integral deformable layer of FIG. 26A including depressions formed into the deformable layer;

FIG. 27 is a perspective view of a typical operating table;

FIG. 28A is a top plan view of the support surface overlay of FIG. 1A, further including a selectively inflatable bolster compartment according to the present disclosure;

FIG. 28B is a top plan view of the support surface overlay of FIG. 28A with the inflatable bolster compartment shown and the first and second inflatable compartments not shown for clarity;

FIG. 28C is a top plan view of the support surface overlay of FIG. 28A with the first inflatable compartment and inflatable bolster compartment not shown for clarity;

FIG. 28D is a top plan view of the support surface overlay of FIG. 28A with the second inflatable compartment and inflatable bolster compartment not shown for clarity;

FIG. 28E is a side elevation view of the support surface overlay of FIGS. 28B and 28C in a deflated state and strapped to a side rail of an underlying operating table in a horizontal position;

FIG. 28F is a side elevation view of the support surface overlay and operating table of FIG. 28E with the support surface overlay in a deflated state and the operating table in an inclined position;

FIG. 28G is a side elevation view of the support surface overlay of FIGS. 28B and 28C in an inflated state and strapped to a side rail of an underlying operating table in a horizontal position;

FIG. 28H is a side elevation view of the support surface overlay and operating table of FIG. 28G with the support surface overlay in a deflated state and the operating table in an inclined position;

FIG. 29A is a top plan view of a support surface overlay similar to the support surface overlay of FIG. 1A and further including an extended tail and tail tabs configured to be folded to form a tail pocket according to the present disclosure;

FIG. 29B is a top plan view of the support surface overlay of FIG. 29A with the extended tail and tail tabs folded to form the tail pocket; and

FIG. 29C is a rotated side elevation view of the support surface overlay of FIG. 29B showing the tail pocket.

### DETAILED DESCRIPTION OF THE DRAWINGS

Terms such as upper, lower, above, below, front, back, head, foot, left, right, inflated, deflated, lift, raise, lower, expand, collapse, and the like as may be used herein should be construed in a relative, rather than absolute, sense unless context clearly dictates otherwise.

The embodiments shown and described herein are illustrative and not limiting. Those skilled in the art would understand how to modify the illustrative embodiments without departure from the scope of the appended claims.

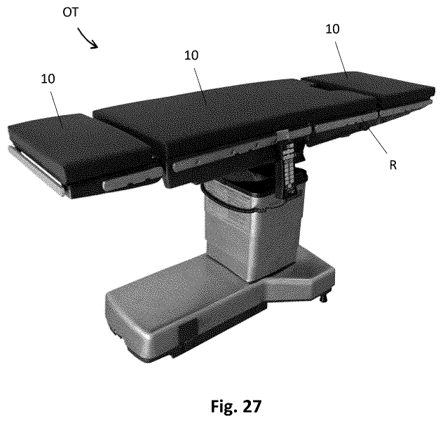

The drawings show illustrative embodiments of a support surface overlay (hereinafter “overlay”) 100 for use with an underlying support surface 10. The underlying support surface 10 may be, for example and without limitation, a pressure redistribution mattress, an upper surface of an operating table, or other surface. For example, FIG. 27 shows an illustrative operating table OT having an upper support surface 10 configured to support a patient, for example, during a surgical procedure. A further support surface 10 in the form of a pressure redistribution mattress is disposed upon the upper surface of the operating table OT, and the overlay 100 is disposed upon the support surface 10, i.e., the pressure redistribution mattress.

The overlay 100 is configured to selectively and simultaneously impart lift and transverse motion to the skin of a user disposed upon the overlay 100. In the course of doing so, the overlay 100 may selectively apply shear forces to, and relieve shear forces from, the skin of a user disposed upon the overlay 100. The overlay 100 may apply such shear forces to the user's skin directly, or through one or more intervening layers, for example, bed sheets, incontinence pads, or the like. Such application and relief of shear forces from the user's skin may enhance local perfusion in the user's skin proximate the locations where the shear forces are applied and relieved.

The overlay 100 includes a first, flat flexible sheet (hereinafter “first sheet”) 102 and a second flat, flexible sheet (hereinafter “second sheet) 104 overlying the first sheet 102. As such, the first sheet 102 may be referred to herein as a lower sheet, and the second sheet 104 may be referred to herein as an upper sheet. The first and second sheets 102, 104 may be made of, for example, polyurethane, PVC, or other joinable materials. The first and second sheets 102, 104 may be of the same thickness or different thicknesses. Each sheet may be of uniform thickness or variable thickness. In embodiments, each of the first and second sheets 102, 104, may have thicknesses in the range 0.005 to 0.015 inches or they may have lesser or greater thicknesses.

The second sheet 104 is joined to the first sheet 102 at predetermined locations by one or more seams 106. The seam(s) 106 may be formed by RF welding, ultrasonic welding, chemical bonding, or any other suitable joining technique. The first sheet 102, the second sheet 104, and the seam(s) 106 cooperate to define to define a bladder having a first selectively inflatable compartment (hereinafter “first compartment”) 108 and a second selectively inflatable compartment (hereinafter “second compartment”) 110. Thus, each of the first compartment 108 and the second compartment 110 is bounded by the first sheet 102, the second sheet 104, and the seam(s) 106.

The seam(s) 106 may define one or more discontinuities in the first sheet 102 and the second sheet 104 such that the first sheet 102 and the second sheet 104 are not completely flat at the seam(s) 106. Notwithstanding, the first sheet 102 and the second sheet 104 may be completely flat at least in some regions defining the first compartment 108 and the second compartment 110 with the overlay 100 in a deflated state, as will be discussed further below. For example, the first sheet 102 and the second sheet 104 may be flat at least in regions defining selectively inflatable pivoting elements and lifting elements, as will be discussed further below.

The first compartment includes a first manifold 112 and a first plurality of selectively inflatable pivoting elements 114 (sometimes referred to herein as “first pivoting elements”) fluidly connected to the first manifold 112. As used herein, the term “pivoting element” refers to a selectively inflatable element defined at least by the first sheet 102, the second sheet 104, and the seam(s) 106 and configured so that, when the overlay 100 is anchored to the support surface 10, at least a portion of the second sheet 104 defining the pivoting element moves in a rotational direction having a first translational component normal to and away from the support surface 10 and a second translational component parallel to the support surface 10 in response to inflation of the corresponding selectively inflatable compartment (and, therefore, the pivoting element) from a deflated state to an inflated state, as will be discussed further below. (The first pivoting elements 114 may further be defined by separation cuts partially decoupling the pivoting elements 114 from adjacent portions of the overlay 100, as will be discussed further below.) Such movement may, but need not, follow an arcuate trajectory. As such, a pivoting element as described herein may impart both lift and transverse motion to a body, for example, a human body, disposed thereon. Similarly, as used herein, the terms “pivot” and “pivoting” refer to the foregoing manner of motion of the pivoting element.

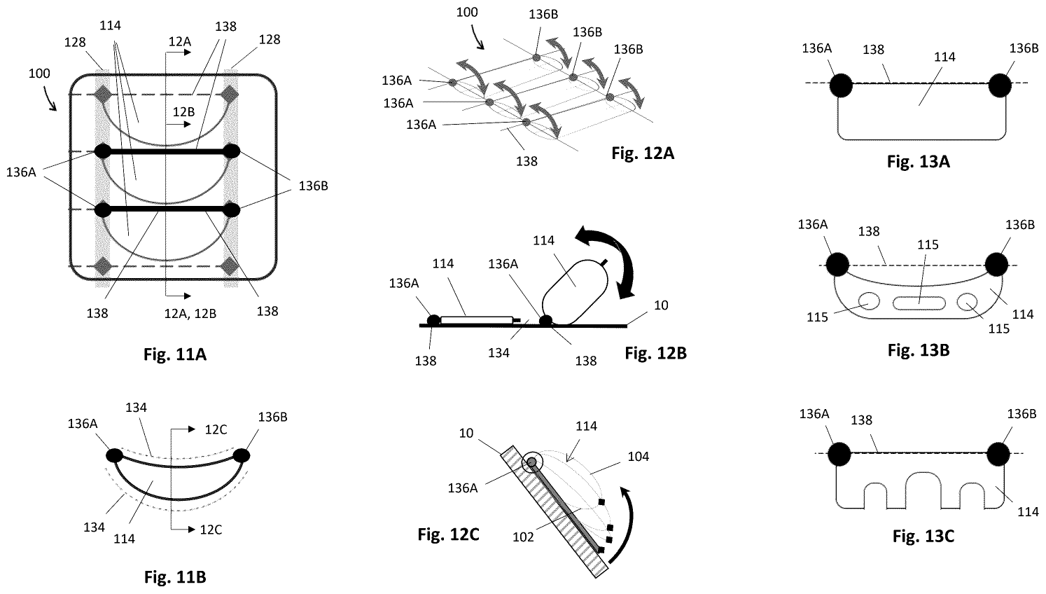

At least ones of the first pivoting elements 114 may be generally elongated, for example, as shown in FIGS. 11A, 11B, 12A, 13A, 13B, and 13C. In embodiments, ones of the first pivoting elements 114 may take other shapes, as desired.

As shown in, for example, FIGS. 11A, 12A, and 12B, ones of the first pivoting elements 114 may pivot in the same rotational direction as others of the first pivoting elements 114. In embodiments, ones of the first pivoting elements 114 may pivot in the opposite rotational direction as others of the first pivoting elements 114 and/or in other rotational directions.

As shown, the first compartment 108 may further include an optional first plurality of selectively inflatable lifting elements 116 (sometimes referred to herein as “first lifting elements”) fluidly connected to the first manifold 112. As used herein, the term “lifting element” refers to a selectively inflatable element defined at least by the first sheet 102, the second sheet 104, and the seam(s) 106 and configured so that, when the overlay 100 is anchored to the support surface 10, at least a portion of the second sheet 104 defining the lifting element moves in a direction generally normal to and away from the support surface 10 in response to inflation of the corresponding selectively inflatable compartment (and, therefore, the lifting element) from a deflated state to an inflated state, as will be discussed further below. (The first lifting elements 116 may further be defined by separation cuts partially decoupling the lifting elements 116 from adjacent portions of the overlay 100, as will be discussed further below.) Such movement may, but need not, follow a purely linear trajectory which may be, but need not be, purely normal to the support surface 10. As such, a lifting element as described herein may predominantly impart lift to a body, for example, a human body, disposed thereon, but may also impart some transverse motion to the body, as well. Similarly, as used herein, the terms “lift” and “lifting” refer to the foregoing manner of motion of the lifting element.

The first compartment 108 may be fluidly connected to a first source of pressurized gas (for example, air) or other fluid (hereinafter “first fluid source”) (not shown) and to a first vent (not shown) via a first conduit 118 fluidly connected to, and extending from, the first manifold 112. The first compartment 108 (including the first manifold 112, the first pivoting elements 114, and the first lifting elements 116) may be selectively inflated to an inflated state by selectively aligning the first conduit 118 with the first fluid source (not shown), and selectively deflated to a deflated state by selectively aligning the first conduit 118 with the first vent (not shown), as would be understood by one skilled in the art. The first conduit 118 may be, but need not be, isolated from the first vent (not shown) when it is connected to the first fluid source (not shown), and vice versa.

Similarly, the second compartment 110 includes a second manifold 120 and a second plurality of selectively inflatable pivoting elements 122 (sometimes referred to herein as “second pivoting elements”) fluidly connected to the second manifold 120. As shown, the second compartment 110 may further include an optional second plurality of lifting elements 124 (sometimes referred to herein as “second lifting elements”) fluidly connected to the second manifold 120. The second pivoting elements 122 and second lifting elements 124 may be, but need not be, structurally and operationally similar to the first pivoting elements 114 and first lifting elements 116.

The second compartment 110 may be fluidly connected to a second source of pressurized gas (for example, air) or other fluid (hereinafter “second fluid source”) (not shown) and to a second vent (not shown) via a second fluid conduit 126 fluidly connected to, and extending from, the second manifold 120. (In embodiments, the first fluid source and the second fluid source may be the same fluid source. As such, the first fluid source and the second fluid source may be interchangeably referred to herein as “the fluid source.”) The second compartment 110 (including the second manifold 120, the second pivoting elements 122, and the second lifting elements 124) may be selectively inflated to an inflated state by selectively aligning the second conduit 126 with the fluid source (not shown), and selectively deflated to a deflated state by selectively aligning the second conduit 126 with the second vent (not shown), as would be understood by one skilled in the art. The second conduit 126 may be, but need not be, isolated from the second vent (not shown) when it is connected to the second fluid source (not shown), and vice versa.

A controller (not shown) may be provided to effect inflation and deflation of the first and second compartments 108, 110. Such a controller may include or be fluidly connected to the fluid source, the vent, the first fluid conduit 118 and the second fluid conduit 126. The controller may include and/or control one or more pumps, valves, and control elements configured to effect selective inflation and deflation of the first and second compartments 108, 110.

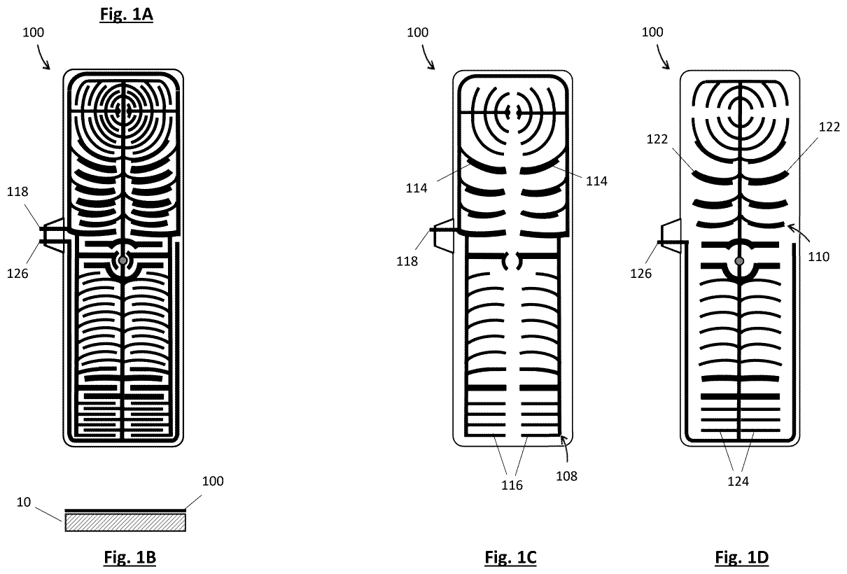

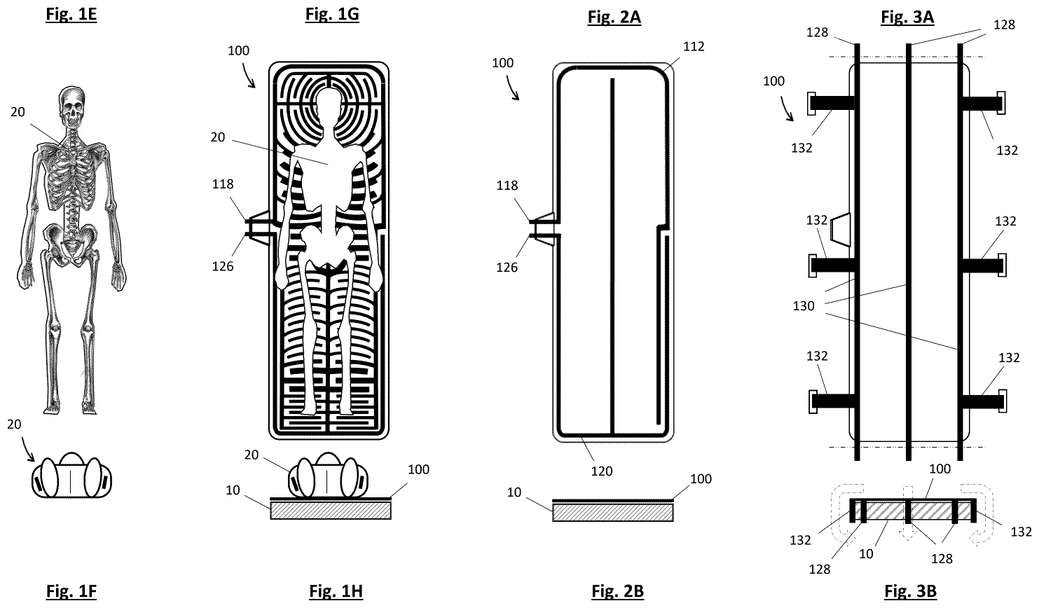

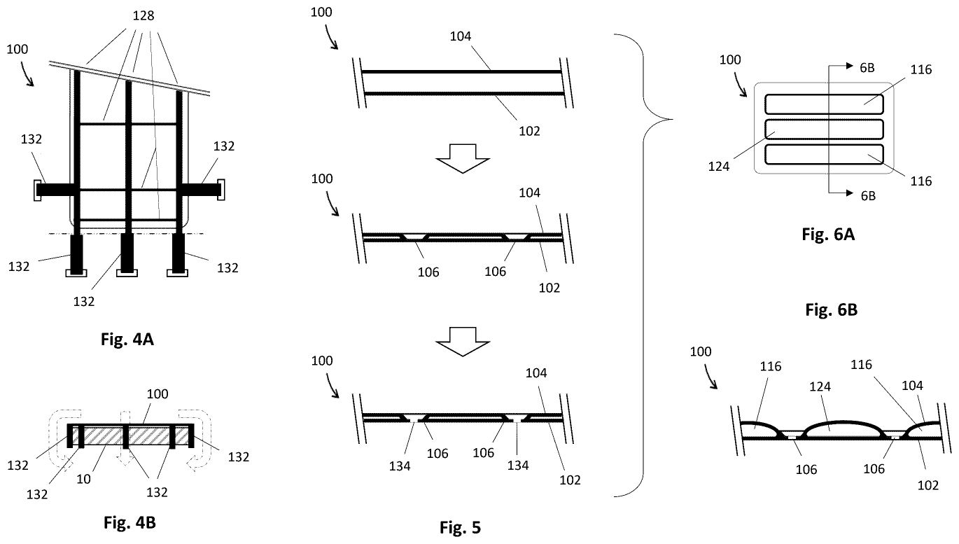

As best shown in FIGS. 1A, 1C, 1D, and 1G, the first pivoting elements 114, first lifting elements 116, second pivoting elements 122, and second lifting elements 124 may be arranged in rows, columns and/or otherwise in a manner that mimics the skeletal structure 20 of a user that may lie upon the overlay 100 during use thereof. These drawings shows ones of the first and second lifting elements 116, 124 cooperatively arranged in circular patterns in regions of the overlay 100 corresponding to the head and sacrum regions of a user that might lie upon the overlay 100. These drawings also shows other ones of the first and second lifting elements 116, 124 cooperatively arranged in linear and curvilinear manners in other regions of the overlay 100. These drawings further shows ones of the first and second pivoting elements 114, 122 arranged in curvilinear manners in regions of the overlay 100 corresponding to the torso of a user that might lie upon the overlay 100. For example, as shown, the first and second pivoting elements 114, 122 may be configured to correspond to the rib or rib cage structure of a user that might lie upon the overlay 100 Also, ones of the first pivoting elements 114 may be interposed between ones of the second pivoting elements 122 and vice versa, and ones of the first lifting elements 116 may be interposed between ones of the second lifting elements 124, and vice versa.

In embodiments, portions of the first and second sheets 102, 104 of the overlay 100 may define one or more anchor zones 128, each anchor zone 128 configured to resist stretching in response to a corresponding tensioning force applied to the overlay in the direction of the anchor zone 128. Such anchor zones 128 may extend in a first direction, for example, from a first end of the overlay 100 to a second end of the overlay 100 opposite the first end of the overlay 100. The first end of the overlay 100 may be referred to herein as the head end of the overlay 100, and the second end of the overlay 100 may be referred to herein as the foot end of the overlay 100. Alternatively or additionally, such anchor zones 128 may extend in a second direction, for example, from a first side of the overlay 100 to a second side of the overlay 100 opposite the side of the overlay 100. The first side of the overlay 100 may be referred to herein as the left side of the overlay 100, and the second side of the overlay 100 may be referred to herein as the right side of the overlay 100. The anchor zones 128 may, but need not, coincide with at least portions of one or both of the first manifold 112 and the second manifold 120.

Typically, the portions of the first and second sheets 102, 104 defining the anchor zones 128 would be free of discontinuities tending to enable elongation of the overlay 100 in response to tensioning forces applied to the overlay 100, as discussed above. For example, as shown in FIGS. 21A-21D, the portions of the first and second sheets 102, 104 defining the anchor zones 128 typically would be free of separation cuts extending through the first and second sheets 102, 104 of the overlay 100, for example, separation cuts decoupling the first pivoting elements 114, first lifting elements 116, second pivoting elements 122, and second lifting elements 124 from adjacent portions of the overlay 100, as will be discussed further below. That is, such separation cuts would not extend into the anchor zones 128.

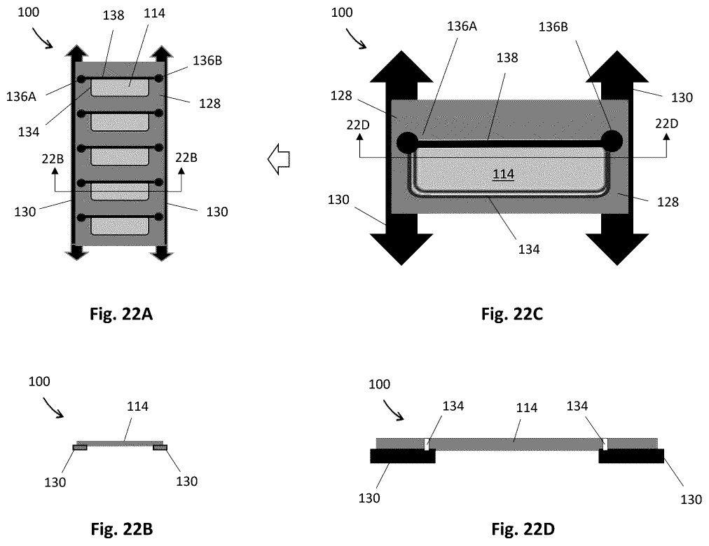

In embodiments, any or all of the anchor zones 128 may include respective anchor zone reinforcements 130, for example, a strap or other layer of additional material underlying the first sheet 102 and joined to at least a portion of the first sheet 102. The anchor zone reinforcement 130, where provided, typically would lessen the propensity of the overlay 100 to stretch in response to tensioning forces applied to the overlay 100 in a direction corresponding to the orientation of the respective anchor zone 128. In embodiments including the anchor zone reinforcements 130, for example, as shown in FIGS. 22A-22D, one or more separation cuts could extend through the first and second sheets 102, 104 of the overlay 100 in an anchor zone 128, but not through the corresponding anchor zone reinforcement 130.

In embodiments, anchor straps 132 (or other mechanical attachment features) may be connected to and extend from the overlay 100 for use in securing the overlay 100 to the underlying support surface 10. Typically, the anchor straps 132 would be located in alignment with and extend from respective ones of the anchor zones 128 and/or the anchor zone reinforcements 130. The anchor straps 132 may be used to place and maintain the overlay 100 in a taut configuration upon the underlying support surface 10, thereby mitigating a tendency for portions of the overlay 100 adjacent the first pivoting elements 114, first lifting elements 116, second pivoting elements 122, and second lifting elements 124 from lifting away from the underlying support surface 10 when the first pivoting elements 114, first lifting elements 116, second pivoting elements 122, and second lifting elements 124 are in the inflated state and/or transition between the deflated and inflated states.

In embodiments, as mentioned above, each of the first pivoting elements 114 and second pivoting elements 122 may be at least partially decoupled from adjacent portions of the overlay 100 by a corresponding separation cut 134 extending through the overlay 100 adjacent and around a corresponding portion of the respective pivoting element 114, 122. Each separation cut 134 may extend through the first and second sheets 102, 104 of the overlay 100 coincident with the seam(s) 106, for example, through the center of the portion of the seam(s) 106, defining (in cooperation with the first and second sheets 102, 104) the respective pivoting element 114, 122. The separation cuts 134 may be made by laser cutting, lancing, or other suitable methods.

Each separation cut 134 thus may at least partially define the shape of the corresponding pivoting element 114, 122. For example, FIG. 11A shows separation cuts 134 defining part-circular pivoting elements 114, 122, while FIG. 11B shows separation cuts 134 defining a banana-shaped pivoting element 114, 122. In embodiments, the separation cuts 134 could be configured to define pivoting elements 114, 122 having other desired shapes, for example without limitation, rectangular, as shown in FIGS. 12A and 13A. In embodiments, the pivoting elements 114, 122 can have more complex configurations, for example, as shown in FIGS. 13B and 13C. FIG. 13B shows a banana-shaped pivoting element 114, 122 having non-inflatable regions 115 therein. FIG. 13C shows a generally rectangular pivoting element 114, 122 modified to define finger-like projections.

Each separation cut 134 extends from a corresponding first anchor point 136A proximate a first of the anchor zones 128 to a second corresponding anchor point 136B proximate a second of the anchor zones 128. As suggested above, the separation cuts 134 typically would not extend into the anchor zones 128. In embodiments including anchor zone reinforcements 130, however, the separation cuts 134 could extend into an anchor zone 128, for example, as discussed above.

The first and second anchor points 136A, 136B cooperate to define a hinge line 138 extending therebetween. The hinge line 138 is located asymmetrically relative to the respective pivoting element 114, 122. As such, the overlay 100 is configured to enable each pivoting element 114, 122 to pivot about the hinge line 138 defined by the first and second anchor points 136A, 136B and with respect to portions of the overlay 100 adjacent the pivoting elements 114, 122. With the overlay 100 disposed upon and anchored to the underlying support surface 10, for example, using the anchor straps 132 as discussed above, this pivoting of the pivoting elements 114, 122 would be substantially limited to pivoting away from the underlying support surface 10.

In embodiments, portions of the either or both of the first sheet 102 and the second sheet 104 defining any of all of the first and second pivoting elements 114, 122 may be configured to stretch to a greater degree than adjacent portions of either or both of the first sheet 102 and second sheet 104 when subjected to a given inflation pressure or range of inflation pressures. In embodiments, this characteristic may be imparted by providing the portion of either or both of the first sheet 102 and second sheet 104 defining any of all of the first and second pivoting elements 114, 122 with multiple thicknesses. In such embodiments, the separation cuts 134 decoupling the first and second pivoting elements 114, 122 from adjacent portions of the overlay 100 may be, but need not be, omitted.

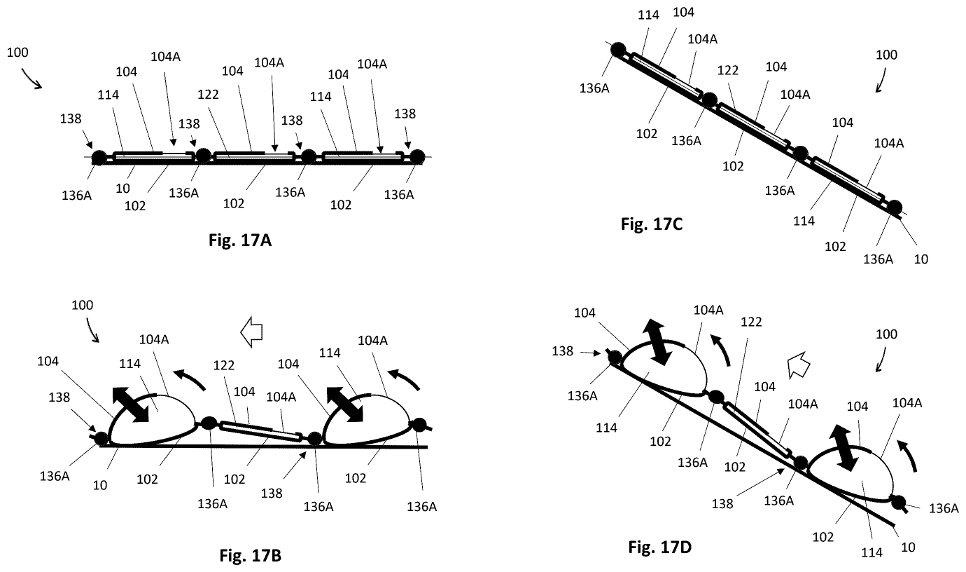

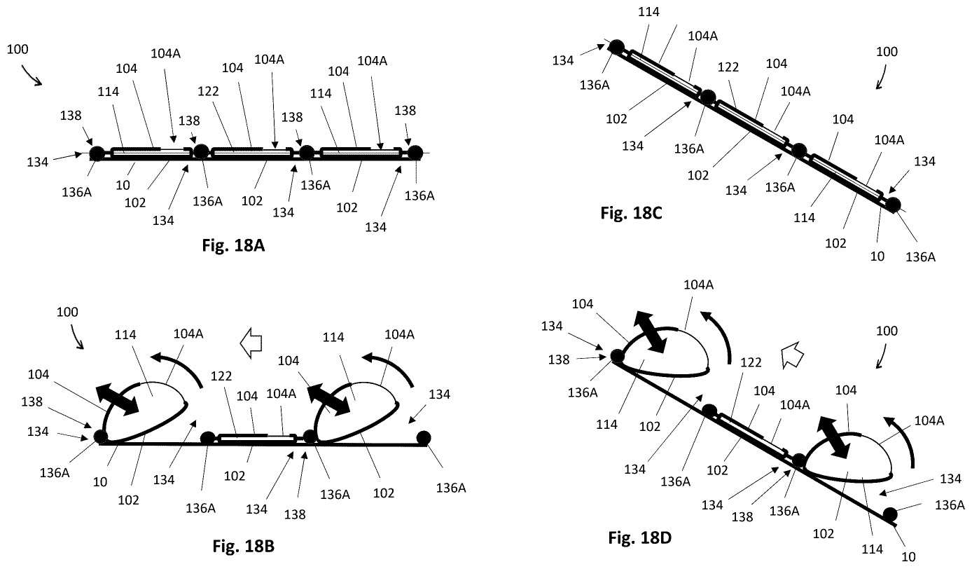

For example, as shown in FIG. 17A-17D, a first portion 104A of the second sheet 104 defining a given pivoting element 114, 122 may be of reduced thickness relative to a second portion of the second sheet 104 defining the given pivoting element 114, 122. Alternatively or additionally, the first sheet 102 could be similarly configured. The portion 104A of reduced thickness stretches to a greater extent than adjacent portions of the second sheet 104 when the respective pivoting element 114, 122 transitions from a deflated state to an inflated state. This phenomenon permits the respective pivoting elements 114, 122 to pivot with respect to the hinge line 138, as discussed above, even when the separation cuts 134 are omitted. FIGS. 18A-18D show a similar embodiment including the separation cuts 134.

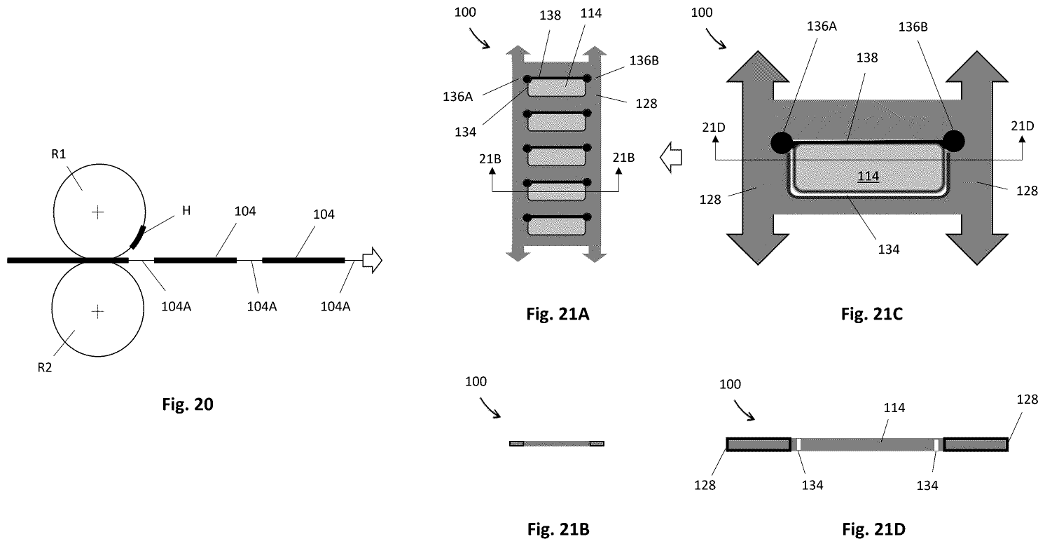

As shown in FIG. 20, the areas of reduced thickness 104A may be created by passing the second sheet 104 of the overlay 100 through a set of rollers R1, R2 including a roller R1 having a high area H proud of adjacent portions of the roller R1, the high area corresponding to the region of reduced thickness of the pivoting element 114, 122, so that the high area H embosses or otherwise locally thins the foregoing first portion of the second sheet 104. Alternatively or additionally, the first sheet 102 could be similarly configured.

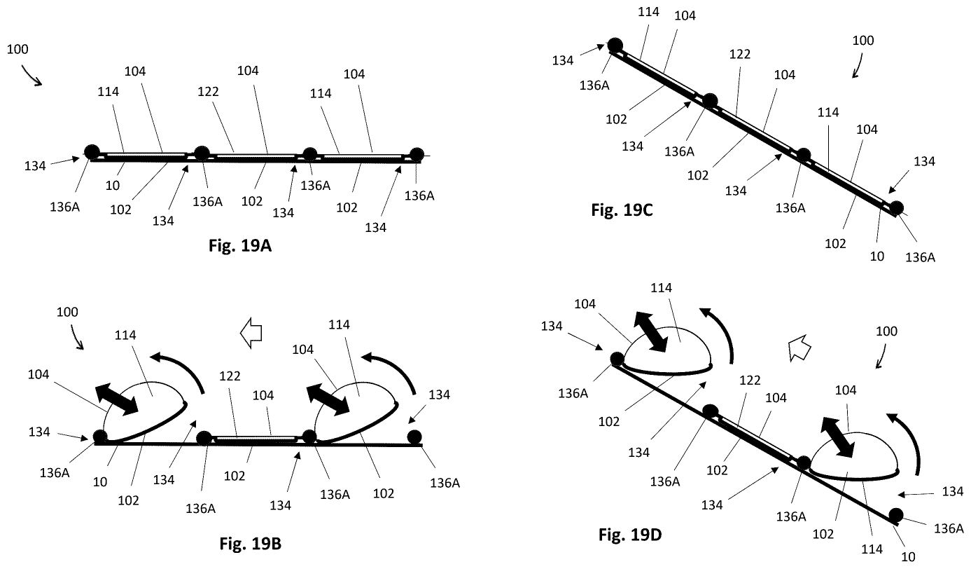

In embodiments, as shown in FIGS. 19A-19D, the second sheet 104 in its entirety may be configured to stretch to a greater degree than the first sheet 102. This configuration could be achieved, for example, by providing the second sheet 104 in a lesser thickness than the first sheet 102 and/or providing the second sheet 104 in a material having different stretch properties than the material of the first sheet 102. Alternatively or additionally, the first sheet 102 in its entirety may be configured to stretch to a greater degree than the second sheet 104 in a similar manner.

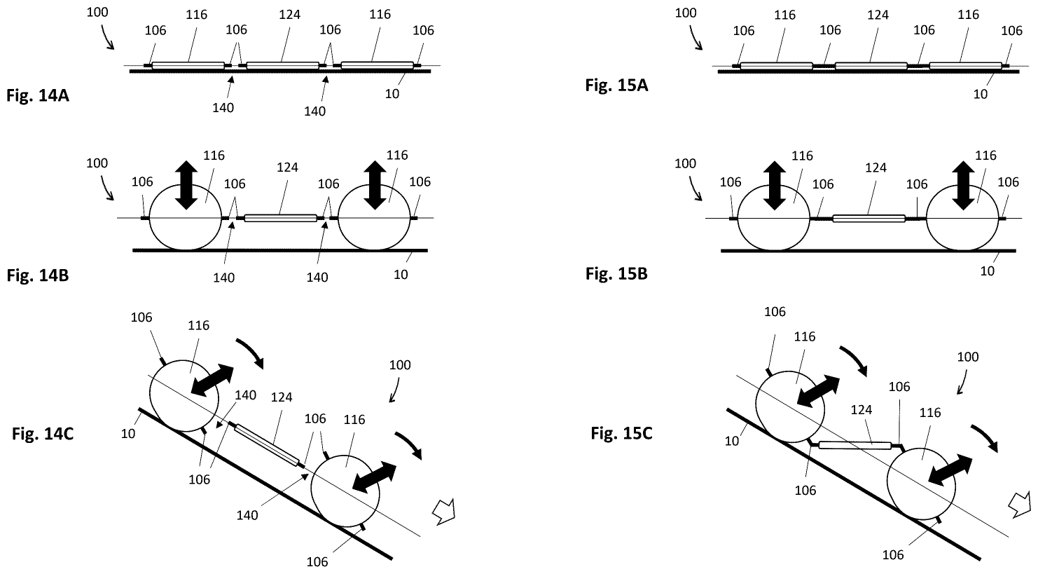

Similarly, as shown in FIGS. 14A-15C, each of the first lifting elements 116 and second lifting elements 124 may be, but need not be, at least partially decoupled from adjacent portions of the overlay 100 by a corresponding separation cut 140 extending through the overlay 100 adjacent and around a corresponding portion of the respective lifting element 116, 124. Each separation cut 140 may extend through the overlay 100 coincident with the seam(s) 106, for example, through the center of the portion of the seam(s) 106, defining (in cooperation with the first and second sheets 102, 104) the respective lifting element 116, 124. The separation cuts 140 may be made by laser cutting, lancing, or other suitable methods.

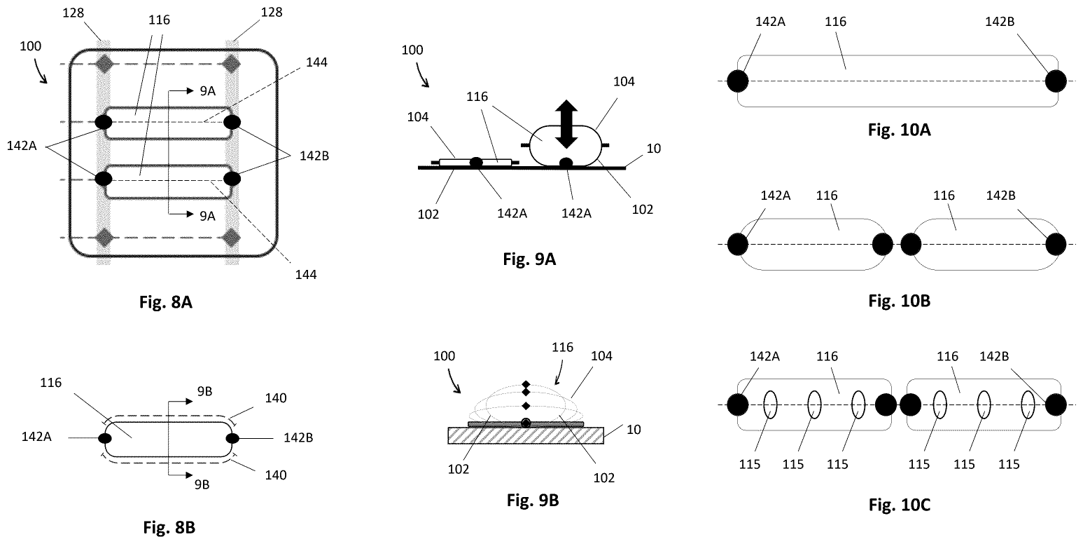

Each separation cut 140 thus may at least partially define the shape of the corresponding lifting element 116, 124. For example, FIGS. 8A and 8B show separation cuts 140 defining generally rectangular lifting elements 116, 124. Also, FIGS. 10A, 10B, and 10C show generally rectangular lifting elements 116, 124 of various sizes. FIG. 10C shows a generally rectangular lifting element having non-inflatable regions 115. In embodiments, the separation cuts 140 could be configured to define lifting elements 116, 124 having other desired shapes.

Each separation cut 140 extends from a corresponding first anchor point 142A proximate a first of the anchor zones 128 to a second corresponding anchor point 142B proximate a second of the anchor zones 128. As suggested above, the separation cuts 140 typically do not extend into the anchor zones 128. In embodiments including anchor zone reinforcements 130, the separation cuts 140 could extend into an anchor zone 128 as discussed above.

The first and second anchor points 142A, 142B cooperate to define an anchor line 144 extending therebetween. The anchor line 144 may be located symmetrically relative to the respective lifting element 116, 124. As such, the overlay 100 is configured to enable each lifting element 116, 124 to expand from the anchor line 144 in a direction transverse to a plane defined by a portion of the overlay 100 adjacent the respective lifting element 116, 124. With the overlay 100 disposed upon and anchored to an underlying support surface 10, for example, using the anchor straps 132 as discussed above, this expansion of the lifting elements 116, 124 would be substantially limited to expansion in a direction away from the underlying support surface 10.

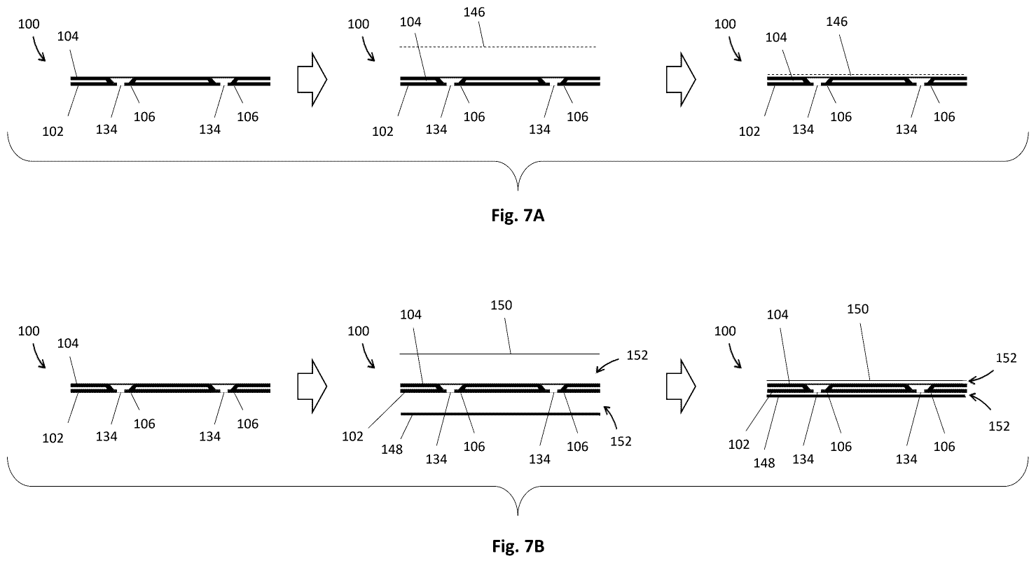

In embodiments, the overlay 100 may include one or more additional material layers attached to or otherwise operably associated with the bladder defined by the first and second sheets 102, 104 and the seam(s) 106. For example, as shown in FIG. 7A, the overlay 100 may include a protective sheet 146 disposed upon the second sheet 104. The protective sheet 146 may be removably attached to the second sheet 104 using an adhesive having sufficient adhesive strength to inhibit unintentional removal, yet weak enough to enable a user to easily remove the tear-off sheet 146 as may be desired. While attached to the overlay 100, the protective sheet 146 may serve to stabilize the decoupled pivoting elements 114, 122 and lifting elements 116, 124 and inhibit their movement relative to adjacent portions of the overlay 100, thereby affording a degree of protection to the overlay 100 generally prior to use. The protective sheet 146 may be removed from the overlay 100 prior to use of the overlay 100, thereby freeing the pivoting elements 114, 122 and lifting elements 116, 124 and enabling them to pivot and lift as discussed above. In embodiments, the protective sheet 146 could be made of a material having weak tensile strength so that the protective sheet ruptures proximate the separation cuts 134, 140 when the overlay 100 is inflated to the inflated state from the deflated state. In such embodiments, the protective sheet 146 may remain attached to the overlay 100 when the overlay 100 is put into use. In embodiments, the protective sheet 146 may be applied to the first sheet 102 in addition to or instead of the second sheet 104.

Alternatively or additionally, the separation cuts 134, 140 may be formed to substantially decouple the pivoting elements 114, 122 and lifting elements 116, 124 from adjacent portions of the overlay 100, yet provide weak, frangible connections (not shown) between the pivoting elements 114, 122 and lifting elements 116, 124 from adjacent portions of the overlay 100 in the regions of the separation cuts 134, 140. These weak, frangible connections (not shown) would be sufficiently strong to maintain a connection between the pivoting elements 114, 122 and lifting elements 116, 124 and the adjacent portions of the overlay 100 prior to use of the overlay 100, yet weak enough to rupture when the overlay 100 is inflated to the inflated state from the deflated state, thereby decoupling the pivoting elements 114, 122 and lifting elements 116, 124 from the adjacent portions of the overlay 100. In such embodiments, the protective sheet 146 may be, but need not be, omitted.

In embodiments, the separation cuts 134, 140 may be formed to substantially decouple the pivoting elements 114, 122 and lifting elements 116, 124 from adjacent portions of the overlay 100, yet provide resilient, durable connections (not shown) between the pivoting elements 114, 122 and lifting elements 116, 124 from adjacent portions of the overlay 100 in the regions of the separation cuts 134, 140. These durable connections (not shown) would be sufficiently strong to maintain a connection between the pivoting elements 114, 122 and lifting elements 116, 124 and the adjacent portions of the overlay 100, even when the overlay 100 is inflated to the inflated state from the deflated state. Such resilient, durable connections may stretch in response to inflation of, and contract in response to deflation of, the respective pivoting elements 114, 122 and lifting elements 116, 124, thus enabling selective pivoting and lifting of the pivoting elements 114, 122 and lifting elements 116, 124, respectively. In such embodiments, the protective sheet 146 may be, but need not be, omitted. In embodiments, such durable connections (not shown) may eventually break, yet the pivoting elements 114, 122 and lifting elements 116, 124 may remain operable.

Also, as shown in FIG. 7B, the overlay 100 may include a first (or lower) envelope sheet 148 underlying the first sheet 102 and may further include a second (or upper) envelope sheet 150 overlying the second sheet 104. The first and second envelope sheets 148, 150 may be joined to either of the first and second sheets 102, 104 using any suitable joining technique, for example, those discussed above in connection with the joining of the first sheet 102 to the second sheet 104. The first and second envelope sheets 148, 150 may cooperate to define an envelope in turn defining an interior region 152 containing at least a portion of the first and second sheets 102, 104, and at least of the positioning elements 114, 122 and lifting elements 116, 124. In embodiments, the second envelope sheet 150 may be omitted and the first envelope sheet 148 may either underlie the first sheet 102 or overlie the second sheet 104.

The first and second envelope sheets 148, 150 may provide protection for the pivoting elements 114, 122 and lifting elements 116, 124 and inhibit inadvertent movement of the pivoting elements 114, 122 and lifting elements 116, 124 or inadvertent rupture of the protective sheet 146 (if provided) during normal handling of the overlay 100, for example, in the course of anchoring the overlay 100 to the support surface 10 or otherwise handling the overlay prior to and/or in preparation for use thereof. The first and second envelope sheets 148, 150 also could facilitate cleaning of the overlay 100 after use.

In embodiments including the second envelope sheet 150, the coefficient of friction between the second sheet 104 and the second envelope sheet 150 may be sufficient to enable the second sheet 104 to apply shear forces to a user's skin via the intervening second envelope sheet 150, as discussed further below.

The overlay 100 may be used as follows. The overlay 100 may be placed upon the underlying support surface 10. The overlay 100 further may be anchored to the support surface 10, for example, using the anchor straps 132, as discussed above.

With the overlay 100 anchored to the support surface 10, the first compartment 108 and the second compartment 110 (and thus the first and second pivoting elements 114, 122 and the first and second lifting elements 116, 124) may be selectively inflated and deflated. In embodiments, the first and second compartments 108, 110 may be selectively inflated to operating pressures in the range of 1.0-6.0 psig or to lesser or greater pressures.

This selective inflation and deflation of the first and second compartments 108, 110 may be alternating, simultaneous, or otherwise. For example, one of the first and second inflatable compartments 108, 110 may be inflated from a deflated state to an inflated state while the other of the first and second compartments 108, 110 is in and remains in a deflated state. Similarly, one of the first and second inflatable compartments 108, 110 may be deflated from an inflated state to a deflated state while the other of the first and second compartments 108, 110 is and remains in an inflated state. Also, both of the first and second compartments 108, 110 can be inflated from a deflated state or deflated from an inflated state simultaneously. Further, one of the first and second compartments 108, 110 can be inflated from a deflated state while the other of the first and second compartments 108, 110 is being deflated from an inflated state. Moreover, each of the first and second inflatable compartments 108, 110, when in the inflated state, may be held in the inflated state for a first predetermined time prior to deflating the respective compartment from the inflated state to the deflated state. Similarly, each of the first and second inflatable compartments 108, 110, when in the deflated state, may be held in the deflated state for a second predetermined time prior to inflating the respective compartment from the deflated state to the inflated state. The first and second predetermined times may be selected as desired and may the same time or different times. As suggested above, a controller (not shown) may be provided and configured to enable the foregoing inflation and deflation of the first and second compartments 108, 110.

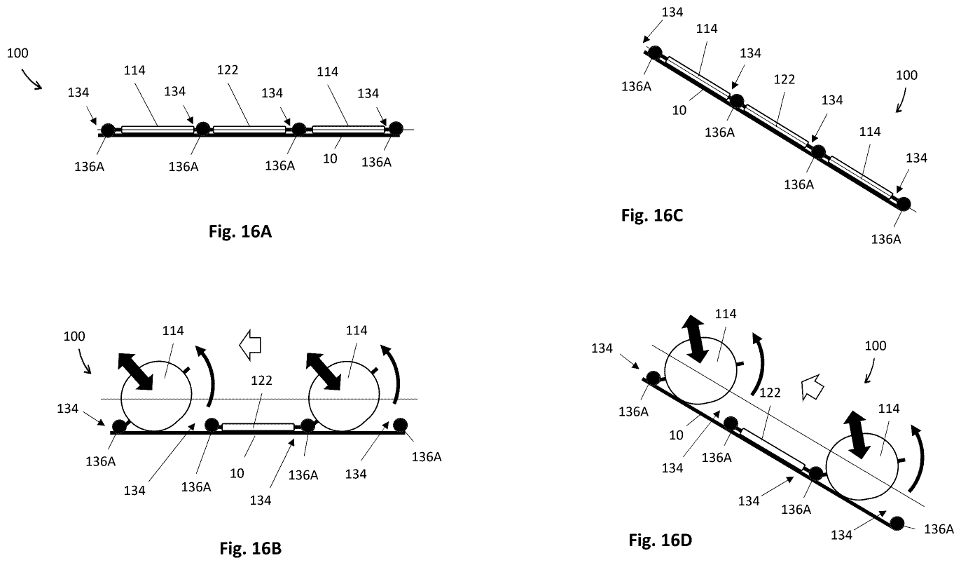

In response to inflation of the first compartment 108 (and thus inflation of the first pivoting elements 114 and first lifting elements 116) from the deflated state to the inflated state, and with the overlay 100 anchored to the support surface 10, each first pivoting element 114 expands and pivots with respect to the hinge line 138 and portions of the overlay 100 adjacent the respective first pivoting element 114 in a direction away from the support surface 10. The pivoting is enabled by the separation cuts 134 and/or the relative stretch characteristics of the first and/or second sheets 102, 104, as discussed above, and as shown in FIGS. 16A-16D and 17A-17D, respectively. The pivoting effect may be enhanced in embodiments including both the separation cuts 134 and first and/or second sheets 102, 104 including regions of reduced thickness, for example, region of reduced thickness 104A, or other enhanced stretch characteristics, as shown in FIGS. 18A-18D

With a user disposed upon the overlay 100, and in response to the pivoting of the first pivoting element 114, the portion of the second sheet 104 defining the respective first pivoting element 114 in contact with the user's skin (either directly or through one or more intervening sheets, for example, the second envelope sheet 150, separate bed sheets (not shown), or incontinence pads (not shown)) may pull the user's skin in the direction in which the first pivoting element 114 pivots. Also, each first lifting element 116 expands in a direction away from the support surface 10 with respect to the respective anchor line 144, thereby increasing contact pressure and providing lift to corresponding portions of the users skin, while relieving contact pressure from adjacent portions of the user's skin.

Deflation of the overlay 100 from the inflated state to the deflated state yields the opposite results, as would be understood by one skilled in the art. More specifically, in response to deflation of the first compartment 108 (and thus deflation of the first pivoting elements 114 and first lifting elements 116) from the inflated state to the deflated state, each first pivoting element 114 collapses and pivots toward the support surface 10 about the respective hinge line 138, thereby relieving tension placed on the user's skin during inflation resulting from pivoting of the first pivoting member 114. Also, each first lifting element 116 collapses toward the support surface 10 with respect to the respective anchor line 144.

The second pivoting elements 122 and second lifting elements 124 respond similarly to inflation and deflation of the second compartment 110, and they may impart forces to a user disposed on the overlay 100 in a similar manner.

As suggested above, the foregoing pivoting of the first and second pivoting elements 114, 122 away from and toward the support surface 10 in response to inflation and deflation of the first and second compartments 108, 110 tends to alternatingly apply and release substantial interface pressure and shear forces to and from corresponding portions of the skin of a patient or other user lying upon the overlay 100 as the overlay 100 is inflated and deflated. Such application of interface pressure and shear forces to the skin may be sufficient to substantially occlude capillary blood flow in the skin proximate the pivoting elements 114, 122, and such release of interface pressure and shear forces from the skin may substantially enable re-establishment of capillary blood flow in the skin proximate the pivoting elements 114, 122. Selective application and release of such interface pressure and shear forces in a predetermined manner may enhance perfusion in the affected portions of the user's skin.

In embodiments, the second compartment 110 and the second conduit 118 may be omitted from the overlay 100 so that the overlay 100 has a single inflatable compartment 108. In such embodiments, the second fluid source (not shown) and the second vent (not shown) also may be omitted. In other embodiments, the overlay 100 may further include one or more selectively inflatable compartments and corresponding conduits addition to the first and second compartments 102, 104. Such embodiments may further include additional corresponding conduits, fluid sources, and vents.

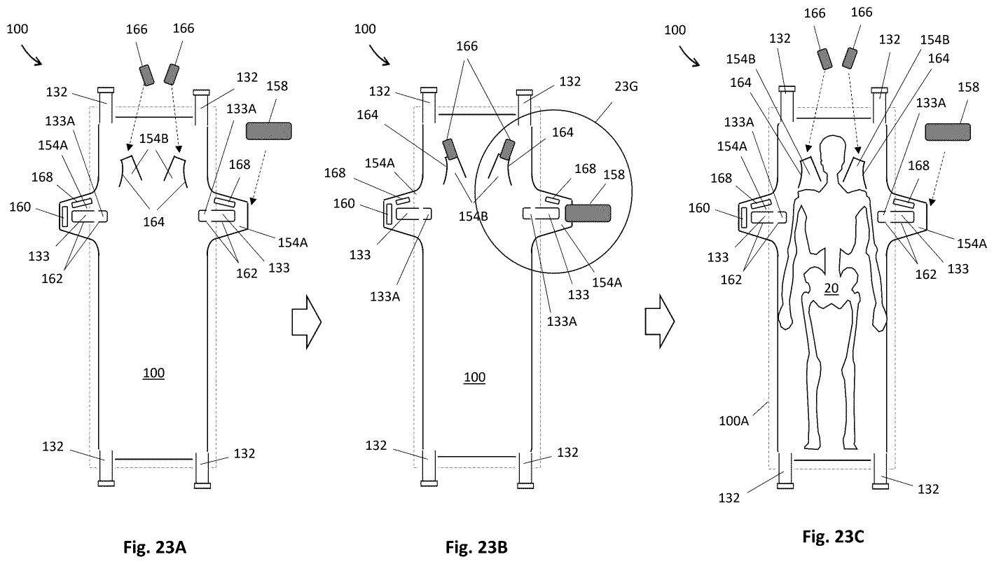

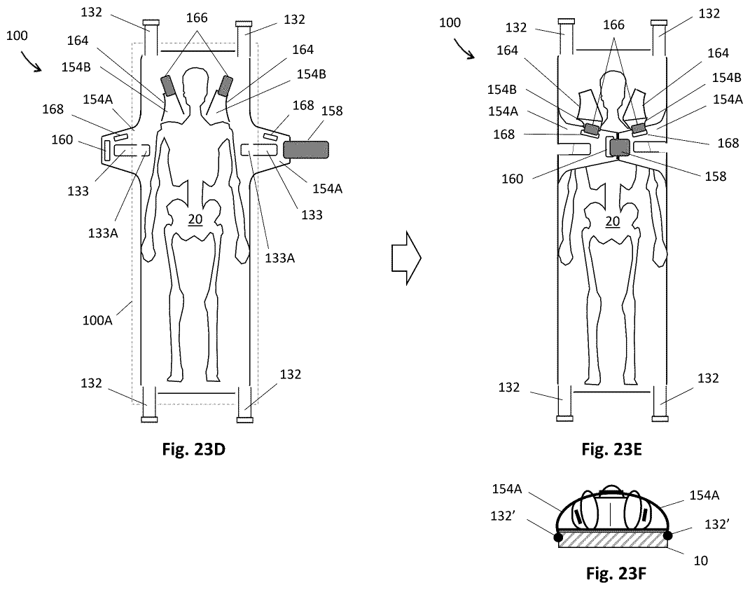

In embodiments, as shown for example in FIGS. 23A-29C, the overlay 100 may be provided with one or more body retention features configured to assist in retaining a user disposed thereon to the overlay 100. Such body retention features may include body retention straps 154 integrated with or separate from the overlay 100. Integrated body retentions straps 154 may be formed monolithically with the bladder of the overlay 100. Alternatively, integrated body retention straps 154 may be formed separately from the bladder and post-attached to the bladder or otherwise to the overlay 100. In embodiments, separately-formed body retention straps 154 may remain structurally independent of the overlay 100, as will be discussed further below. The body retention straps 154 may include pivoting and/or lifting elements similar to those described above, as will be discussed further below. Additionally or alternatively, such body retention features may include an integral deformable layer 156 made of a suitable flexible and resilient material and which may have pressure redistribution characteristics, as will be discussed further below. Additionally or alternatively, such body retention features may include a selectively inflatable bolster integrated with the bladder, as will be discussed further below.

FIGS. 23A-23G show an illustrative overlay 100 including one or more body retention straps 154 including upper body straps 154A and shoulder straps 154B integrated with and formed monolithically with a central portion 100A of the overlay 100. FIGS. 23C-23F also show an illustrative human skeletal structure 20 disposed upon the central portion 100A of the overlay 100, as will be discussed further below. FIGS. 23C-23F further show, collectively, an illustrative sequence of using the body retention straps 154. FIGS. 23D-23F similarly show, collectively, an illustrative sequence of using the body retention straps 154. FIG. 23G shows selectively inflatable strap pivoting elements and strap lifting elements disposed in ones of the body retention straps 154, as will be discussed further below.

As best shown in FIGS. 23A-23F, the upper body straps 154A may be embodied as tabs of material of the overlay 100 extending laterally from left and right sides of the central portion 100A of the overlay 100. As shown, the upper body straps 154A may be monolithically formed with one or more of the sheets (that is, any or all of the first and second sheets 102, 104 and the optional first and second envelope sheets 148, 150) defining the overlay 100. An upper body strap securement system may be provided to secure each of the upper body straps 154A to another portion of the overlay 100, for example to the other of the upper body straps 154A. As shown, the upper body strap securement system includes a Velcro strap 158 connected to and extending from one of the upper body straps 154A, in combination with a corresponding slot 160 defined by the other of the upper body straps 154A and configured to receive the free end of the Velcro strap 158.

In use, the upper body straps 154A may be folded out-of-plane from the central portion 100A of the overlay 100 and around the upper body of a user, for example, as represented by skeletal structure 20, disposed thereon. As shown, the upper body straps 154A may be folded around both a user's chest and arms and thereby secure both the user's chest and arms. In embodiments, the upper body straps may be folded around only the user's chest to thereby secure the user's chest, but not arms. In embodiments, one of the upper body straps 154A may be folded around the user's chest and a corresponding one of the user's arms, and the other of the upper body straps may be folded around the user's chest but not around a corresponding other one of the user's arms, thereby securing the user's chest and one of the user's arms. The Velcro strap 158 may be threaded through the slot 160 and folded back upon and adhered to itself to secure the upper body straps 154A to each other and against the user's chest. Although shown as Velcro straps and corresponding slots, the foregoing upper body strap securement system may take any other suitable form, for example without limitation, a belt and buckle and corresponding slot, a snap system, a button and buttonhole system, as would be recognized by one skilled in the art. In embodiments, the upper body straps 154A may be folded over a user's arms and under the user's torso to thereby secure the user's arms.

As also best shown in FIGS. 23A-23F, retention straps 133 may be embodied as tabs extending laterally from left and right sides of the central portion 100A of the overlay 100. For example, the retention straps 133 may be monolithically formed with the upper body straps 154A and then separated therefrom via cuts 162. In use, each of the retention straps 133 may be folded out-of-plane from the central portion 100A of the overlay 100 and around an anchor point of the underlying support surface 10, for example, a side rail R of an operating table OT as shown in FIG. 27.

Alternatively, either or both of the retention straps 133 may be wrapped around the corresponding arm of a user, for example, as represented by skeletal structure 20, disposed thereon, and tucked under another part of the user's body to thereby retain the user's arm to the overlay 100 or to the underlying support structure 10. In embodiments, optional auxiliary retention straps 133A may be formed in a similar manner as the retention straps 133. In such embodiments, a retention strap securement system (not shown) may be provided to secure either or both of the retention straps 133 to the corresponding optional auxiliary retention strap 133A. Such a retention strap securement system may be similar to the upper body strap securement system described above in connection with the upper body straps 154A.

As further best shown in FIGS. 23A-23F, the shoulder straps 154B may be embodied as shoulder flaps monolithically formed with the central portion 100A of the overlay and then separated therefrom via cuts 164. A should strap securement system may be provided to secure each of the shoulder straps 154B to another portion of the overlay 100, for example to the a corresponding one of the upper body straps 154A. As shown, the shoulder strap securement system includes a Velcro strap 166 connected to and extending from each of the shoulder straps 154B, in combination with a corresponding slot 168 defined by a corresponding one of the upper body straps 154A and configured to receive the free end of the Velcro strap 166. In use, the shoulder straps 154B may be folded out-of-plane from the central portion 100A of the overlay 100 and around the shoulders of a user, for example, as represented by skeletal structure 20, disposed thereon. The Velcro strap 166 may be threaded through the slot 168 and folded back upon and adhered to itself to secure the shoulder straps 154B to the upper body straps 154A and against the user's shoulders. Although shown as Velcro straps and corresponding slots, the foregoing shoulder strap securement system may take any other suitable form, for example without limitation, a belt and buckle and corresponding slot, a snap system, a button and buttonhole system, or ties manually, as would be recognized by one skilled in the art.

In embodiments, the overlay 100 need not include any or all of the upper body straps 154A, retention straps 133, and shoulder straps 154B, and could alternatively or additionally include other body retention straps configured to retain other parts of a user's body to the overlay 100. For example, without limitation, the overlay 100 could include straps similar to the upper body straps 154A and/or retention straps 133 configured to retain a user's waist and/or legs to the overlay 100.

As best shown in FIG. 23H, any or all of the body retention straps 154 may, but need not, include corresponding, selectively inflatable, strap pivoting elements 170 and corresponding, selectively inflatable, strap lifting elements 172. Such strap pivoting elements 170 and strap lifting elements 172 may be, but need not be, similar in construction and operation to the first and second pivoting elements 114, 122 and the first and second lifting elements 116, 124, respectively, as discussed further above.

Ones of the strap pivoting elements 170 and strap lifting elements 172 may be fluidly coupled to one of the first and second manifolds 112, 120 otherwise to one of the first and second compartments 108, 110. Similarly, others of the strap pivoting elements 170 and strap lifting elements 172 may be fluidly coupled to the other one of the first and second manifolds 112, 120 or otherwise to the other one of the first and second compartments 108, 110. Such fluid couplings between the strap pivoting elements 170 and strap lifting elements 172 may be defined by the first and second sheets 102, 104 of the overlay 100, for example, as shown in FIG. 23H. Alternatively, such fluid couplings between the strap pivoting elements 170 and strap lifting elements 172 may be embodied via fluid lines external to the overlay 100 (not shown). As such, ones of the strap pivoting elements 170 and strap lifting elements 172 may be selectively inflated and deflated in concert with the first compartment 108 and the first pivoting elements 114 and the first lifting elements 122 thereof, while others of the strap pivoting elements 170 and strap lifting elements 172 may be selectively inflated and deflated in concert with the second compartment 110 and the second pivoting elements 114 and the second lifting elements 124 thereof.

In embodiments, ones of the strap pivoting elements 170 and the strap lifting elements 172 may be selectively inflated and deflated independently from either or both of the first and second compartments 108, 110, and any or all of the first and second pivoting elements 114, 122 and the first and second lifting elements 116, 124 thereof. In such embodiments, the strap pivoting elements 170 and strap lifting elements 172 may be fluidly coupled to a corresponding control system (not shown) effecting such selective inflation and deflation. Such fluid couplings between the strap pivoting elements 170, the strap lifting elements 172, and the control system (not shown) may be internal to or external to the overlay 100, as discussed further above. For example, such fluid coupling may be embodied as a corresponding selectively inflatable compartment or a corresponding manifold defined by the first and second sheets 102, 104 of the overlay 100. In embodiments, such fluid couplings may be via fluid conduits external to the first and second sheets 102, 104 of the overlay 100. In embodiments, the control system (not shown) effecting selective inflation and deflation of the strap pivoting elements 170 and strap lifting elements 172 may also independently effect selective inflation and deflation of the first and second compartments 108, 110. In other embodiments, the control system (not shown) effecting selective inflation and deflation of the strap pivoting elements 170 and the strap lifting elements 172 may be a different control system (not shown) than the control system (not shown) effecting selective inflation and deflation of the first and second compartments 108, 110.

In embodiments, as mentioned above, the body retention straps 154 may be formed separately from the overlay 100. Such separately-formed body retention straps 154 may be, but need not be, post-integrated with the bladder or otherwise with the overlay 100 using any suitable technique, including without limitation stitching or any of the means discussed above for joining the sheets to each one another. The separately-formed body retention straps 154 may include strap pivoting elements 170 and strap lifting elements 172. Such strap pivoting elements 170 and strap lifting elements 172 may be fluidly connected to the overlay 100 or otherwise to a controller configured to selectively inflate and deflate the strap pivoting elements 170 and the strap lifting elements 172, for example, as discussed above in connection with FIGS. 23A-23H.

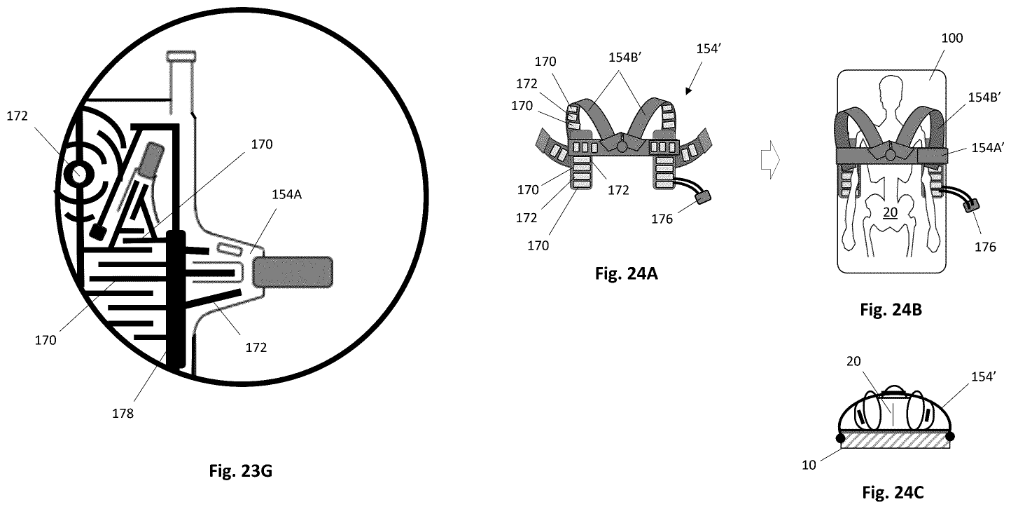

For example, FIG. 24A shows illustrative separately-formed and structurally independent body retention straps 154 embodied as a body retention strap apparatus 154′ including an upper body strap portion 154A′ and a shoulder strap portion 154B′. As shown, each of the upper body strap portion 154A′ and the shoulder strap portion 154B′ includes one or more strap pivoting elements 170 and strap lifting elements 172. Additionally or alternatively, the body retention strap apparatus 154′ may include a retention strap portion (not shown). FIGS. 24B and 24C show the separately-formed and structurally independent body retention strap 154 in use, strapping the chest and shoulders of the skeletal structure 20 to an underlying support surface 10. As shown, the body retention strap apparatus 154′ may include a connector 176 configured to fluidly connect the strap pivoting elements 170 and strap lifting elements 172 of the body retention strap apparatus 154′ to the overlay 100 or otherwise to a controller.

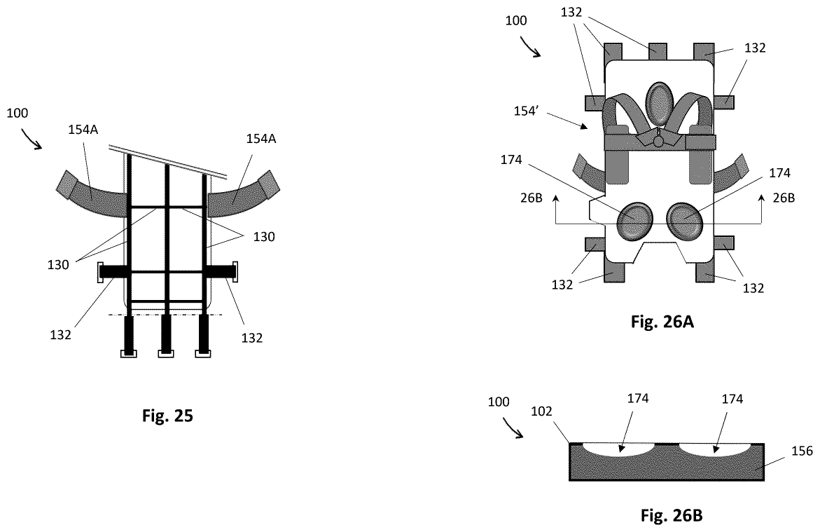

In embodiments, as mentioned above, the separately-formed body retention straps 154 may be post-integrated with the overlay 100. In embodiments, the separately-formed body retention straps 154 may be joined to one or more of the anchor zone reinforcements 130 or anchor straps 132 using any suitable technique, including without limitation any of the techniques discussed above for joining the anchor straps 132 to the sheets of the overlay 100, as discussed above. For example, FIG. 25 shows separately-formed upper body straps 154A post-attached to respective ones of the anchor zone reinforcements 130. In embodiments, the body retention straps 154 may be monolithically formed with the anchor zone reinforcements 130 or anchor straps 132. The combination of the body retention straps 154 with the anchor zone reinforcements 130 or anchor straps 132 may then be post-integrated with the overlay 100.

In embodiments, the separately-formed body retention straps 154 may be assembled to the overlay 100 by mechanical means, for example, by threading the separately-formed body retention straps 154 through corresponding slots (not shown) formed into the overlay 100 and securing the free ends of the separately-formed body retention straps 154 to other portions thereof.

As mentioned above, the overlay 100 may include a retention feature embodied as an integral, flexible and resilient deformable layer 156 layer, for example, as shown in FIGS. 26A and 26B. The deformable layer 156 may be made of any suitable flexible and resilient material of any suitable thickness. For example, without limitation, the deformable layer 156 may be made of open or closed cell foam having a thickness in the range 0.25 inch-2.5 inches or a greater or lesser thickness. The deformable layer 156 is configure to deform in response to the weight of a body disposed thereon and to conform to the contour of the body. As such, a body disposed on the deformable layer 156 may become immersed in the redistribution layer 156. Consequently, the deformable layer 156 may at least partially envelope the body immersed in the deformable layer 156. Such immersion and envelopment tend to effect retention of the body by hindering the body from slipping across the surface of the overlay 100 at the interface of the overlay 100 with the body, even if the body and the overlay 100 are in an inclined orientation. In embodiment, as shown in FIGS. 26A and 26B, the deformable layer 156 may, but need not, include pre-formed depressions 174. Pre-formed depressions 174 may provide further immersion and envelopment of a body disposed on the deformable layer 156 and thus may further effect retention of the body by hindering the body from slipping across the surface of the overlay 100 at the interface of the overlay 100 with the body, even if the body and the overlay 100 are in an inclined orientation.

The deformable layer 156 may connected to the central portion 100A of the overlay 100 and underlie at least a portion of the overlay 100. For example, the deformable layer 156 may underlie and be connected to the first sheet 102 or the first envelope sheet 148. In embodiments, the deformable layer 156 may overlie and be connected to the central portion 100A of the overlay 100. For example, the deformable layer 156 may overlie and be connected to the second sheet 104 or the second envelope sheet 150.

In embodiments including the body retention straps 154, the deformable layer 156 may additionally or alternatively be integrated with the body retention straps 154. In such embodiments, the deformable layer 156 may be connected to the body retention straps 154 on either or both of surfaces thereof facing the user when in use and opposite the user when in use. For example, as shown in FIG. 26A, the deformable layer 156 may be integrated with an overlay 100 and used in connection with the body retention strap apparatus 154′. In embodiments, the deformable layer 156 could be integrated with an overlay 100 having separately-formed and post-integrated body retention straps 154 or with an overlay 100 having monolithically-formed body retention straps 154.

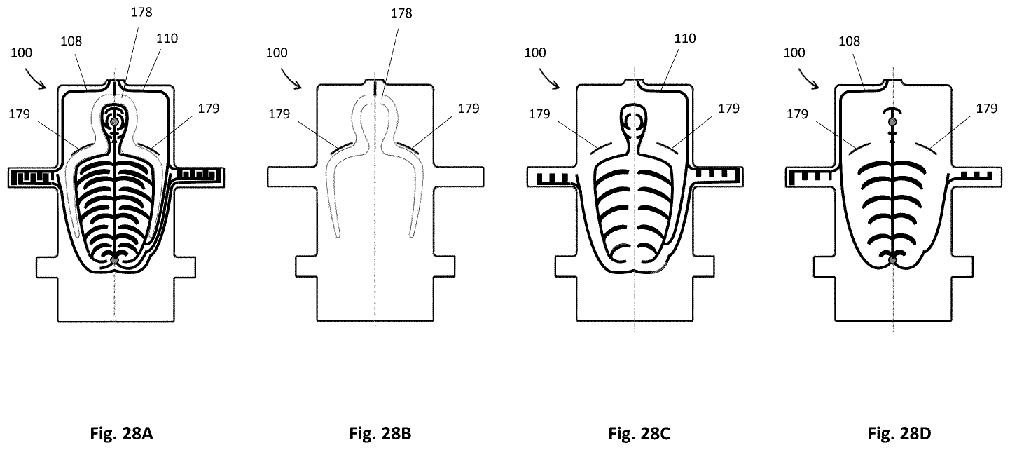

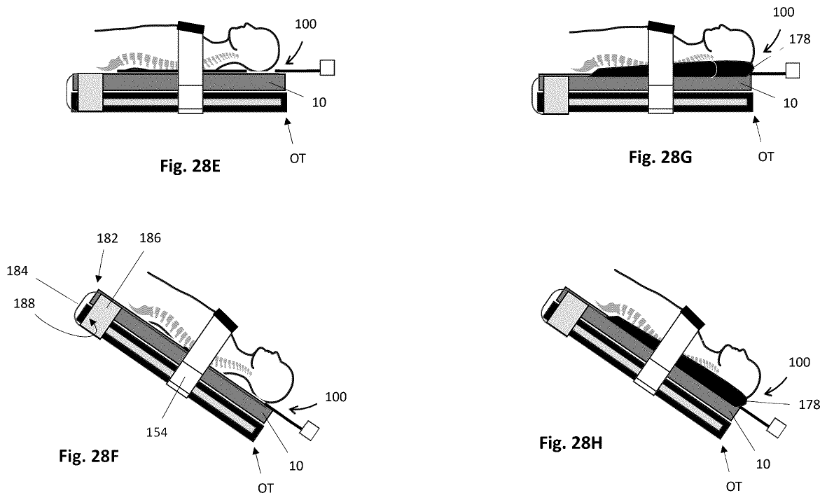

As mentioned above, the overlay 100 may include a retention feature embodied as a selectively inflatable bolster compartment 178 integrated with the bladder, for example, as shown FIGS. 28A-28H. As shown, the bolster compartment 178 has a form complementary to the anatomical form of the torso and shoulder region of a user disposed upon the overlay 100. When inflated, the bolster compartment 178 may at least partially envelope the torso and shoulder region of the user. As such, the inflated bolster compartment 178 may resist sliding of the user across the overlay 100 in a side-to-side direction. The inflated bolster compartment 178 may also resist sliding of the user along the overlay 100 in a foot-to-head direction, especially when the overlay 100 is used on an inclined surface having a foot region thereof elevated with respect to a head region thereof, for example, as shown in FIG. 28H. In embodiments, the inflatable bolster 178 may extend upwardly from the underlying support surface 10 by a distance of about 2 inches or between 1 and 3 inches or a greater or lesser distance. In embodiments, the bolster compartment 178 may be located elsewhere, have a different shape, and be configured to at least partially envelope and restrict movement of another portion of the user's body. In embodiments, a bolster compartment 178 may be provided in a body retention strap 154. In embodiments, more than one bolster compartment 178 may be provided. Each such bolster compartment 178 may be configured to at least partially envelope and restrict movement of a respective portion of the user's body. In embodiments, one or more separation cuts 179 may be provided proximate the bolster compartment 178 to facilitate vertical expansion (lift) of the bolster compartment with respect to adjacent portions of the overlay 100. The bolster compartment 178 may be fluidly connected to a corresponding source of pressurized gas (for example, air) or other fluid. The bolster compartment 178 may be selectively inflated and deflated as desired. In embodiments, the bolster compartment 178 may be inflated and remain inflated for the duration of an event, for example, a surgical event.

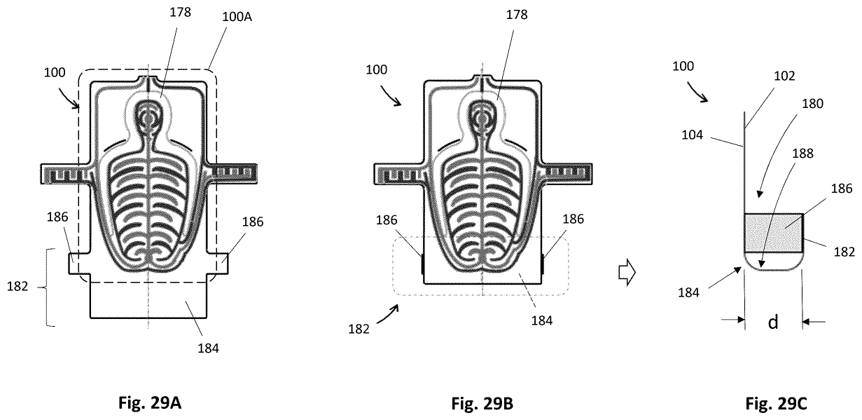

FIG. 29A shows an overlay 100 configured with a tail region 182 including an extended tail 184 and tail tabs 186 configured to be folded and joined together to thereby form a tail pocket 180. FIGS. 29B and 29C show the overlay of FIG. 29A with the extended tail 184 folded so that a portion of the extended tail 184 is doubled back along and spaced from the central portion 100A of the overlay 100. FIGS. 29B and 29C also show the tail tabs 186 folded in a similar manner. FIG. 29C further shows the tail tabs 186 joined to the extended tail 184, thereby defining the tail pocket 180. The tail pocket 180 may be configured to have a height d selected to be at least nominally greater than the thickness of a portion of the corresponding end of an underlying support surface 10, for example, the operating table OT. This configuration allows the pocket 180 to be slipped over the corresponding end of the underlying support surface, for example the operating table OT as shown in FIG. 29D. In use, with the operating table OT and/or support surface 10 in an inclined position with the foot end thereof elevated with respect to the head end thereof, a frictional force imparted to the overlay 100 by the weight of a user disposed upon the overlay 100 tends to pull the overlay 100 in a foot-to-head direction along the upper surface of the operating table OT or the intervening support surface 10. This frictional force tends to cause the bottom 188 of the pocket 180 to engage with the corresponding end of the operating table OT or support surface 10, thereby retaining the tail region 182 of the overlay 100 to the operating table OT or support surface 10 and generally placing the overlay 100 in tension in a foot-to-head direction. Such tensioning of the overlay 100 may enhance the operability of the pivoting elements and lifting elements discussed in further detail above.

1. A support surface overlay comprising:
a bladder, the bladder including:
a first flat, flexible sheet;
a second flat, flexible sheet overlying the first flat, flexible sheet; and
a seam joining the first, flat flexible sheet to the second flat, flexible sheet at predetermined locations; and
a body retention strap operably associated with the bladder, the body retention strap including a selectively inflatable strap pivoting element,
wherein the selectively inflatable strap pivoting element is inflatable and deflatable between a deflated state and an inflated state, and
wherein the selectively inflatable strap pivoting element is configured to pivot relative to adjacent portions of the body retention strap in response to transition of the strap pivoting element between an deflated state and an inflated state.

2. The support surface overlay of claim 1 wherein the body retention strap is configured to wrap around at least a portion of a body received upon the overlay.

3. The support surface overlay of claim 1, wherein the body retention strap is an upper body strap configured to wrap around at least a portion of an upper portion of a body received upon the overlay.

4. The support surface overlay of claim 1, wherein the body retention strap is a shoulder strap configured to wrap around at least a portion of a shoulder of a body received upon the overlay.

5. The support surface overlay of claim 1, wherein the body retention strap is configured to wrap around at least a portion of an arm of a body received upon the overlay or an underlying support surface.

6. The support surface overlay of claim 1 wherein the body retention strap is monolithically formed with a portion of the support surface overlay.

7. The support surface overlay of claim 6 wherein the body retention strap is monolithically formed with the bladder.

8. The support surface overlay of claim 7 wherein the strap pivoting element is fluidly connected to the first selectively inflatable compartment via a fluid conduit internal to the bladder.

9. The support surface overlay of claim 7 wherein the strap pivoting element is fluidly connected to the first selectively inflatable compartment via a fluid conduit external to the bladder.

10. The support surface overlay of claim 1 wherein the body retention strap pivoting element is formed separately from the bladder.

11. The support surface overlay of claim 10 wherein the strap pivoting element is fluidly connected to the first inflatable compartment.

12. The support surface overlay of claim 1 further comprising a flexible and resilient deformable layer integrated with the bladder, wherein the deformable layer is configured to flexibly and resiliently deform under the weight of a body disposed upon the overlay.

13. The support surface overlay of claim 12 wherein the deformable layer defines at least one preformed depression.

14. The support surface overlay of claim 1 further comprising a selectively inflatable bolster compartment configured to at least partially envelope a region of a body of a user disposed upon the support surface overlay.

15. The support surface overlay of claim 14 wherein the region of the user comprises a shoulder region or upper body region.

16. The support surface overlay of claim 14 further comprising a separation cut proximate the selectively inflatable bolster compartment, wherein the separation cut is configured to lift of the selectively inflatable bolster compartment with respect to adjacent portions of the support surface overlay.

17. A body retention strap for use in connection with the support surface overlay of claim 1, the body retention strap comprising a selectively inflatable strap pivoting element,
wherein the selectively inflatable strap pivoting element is inflatable and deflatable between a deflated state and an inflated state, and
wherein the selectively inflatable strap pivoting element is configured to pivot relative to adjacent portions of the body retention strap in response to transition of the strap pivoting element between an deflated state and an inflated state.

18. A support surface overlay system comprising:
a bladder, the bladder including:
a first flat, flexible sheet;
a second flat, flexible sheet overlying the first flat, flexible sheet; and
a seam joining the first, flat flexible sheet to the second flat, flexible sheet at predetermined locations; and
a flexible and resilient deformable layer integrated with the bladder,
wherein the deformable layer is configured to flexibly and resiliently deform under the weight of a body disposed upon the overlay.

19. The support surface overlay system of claim 18 wherein the deformable layer defines at least one preformed depression.

20. A body retention strap for use in connection with a support surface overlay, the body retention strap comprising a selectively inflatable strap pivoting element,
wherein the selectively inflatable strap pivoting element is inflatable and deflatable between a deflated state and an inflated state, and
wherein the selectively inflatable strap pivoting element is configured to pivot relative to adjacent portions of the body retention strap in response to transition of the strap pivoting element between an deflated state and an inflated state.
